(19) United States
(12) Patent Application Publication
(10) Pub. No.: US 2025/0312487 A1
(43) Pub. Date: Oct. 9, 2025

(54) ADENO-ASSOCIATED VIRUS CAPSIDS

(71) Applicant: CHILDREN'S MEDICAL RESEARCH INSTITUTE, Westmead, New South Wales (AU)

(72) Inventors: Adrian WESTHAUS, New South Wales (AU); Leszek LISOWSKI, New South Wales (AU)

(21) Appl. No.: 18/865,267

(22) PCT Filed: May 12, 2023
(86) PCT No.: PCT/AU2023/050398

§ 371 Date: Nov. 12, 2024

(30) Foreign Application Priority Data
May 13, 2022 (AU) 2022901281

(51) Int. Cl. A61K 48/00; A61K 40/11; A61K 40/31; C07K 14/005; C12N 9/22; C12N 15/11; C12N 15/86

(52) U.S. Cl. CPC A61K 48/005; A61K 40/11; A61K 40/31; C07K 14/005; C12N 9/224; C12N 9/226; C12N 15/111; C12N 15/86; C12N 2310/20; C12N 2750/14122; C12N 2750/14143

(57) ABSTRACT

Provided herein are AAV capsid polypeptides comprising peptide modifications relative to the wild-type AAV6 polypeptide that, when present in the capsid of an AAV vector, can facilitate homology directed repair (HDR)-mediated gene editing of human T cells. Also provided are AAV vectors comprising the capsid polypeptides, nucleic acid vectors comprising the encoding nucleic acid molecules, and host cells comprising the vectors, as well as methods of use of such AAV vectors, nucleic acid vectors and host cells.

### FIELD OF THE DISCLOSURE

The present disclosure relates generally to adeno-associated virus (AAV) capsid polypeptides and encoding nucleic acid molecules comprising novel cap genes suitable for vectorization and subsequent use in gene editing, e.g., homology directed repair (HDR)-mediated gene editing of T-cells. The disclosure also relates to AAV vectors comprising the capsid polypeptides, and nucleic acid vectors (e.g., plasmids) comprising the encoding nucleic acid molecules, as well as host cells comprising the vectors. The disclosure also relates to methods and uses of the polypeptides, encoding nucleic acid molecules, vectors and host cells.

### BACKGROUND OF THE DISCLOSURE

Gene therapy and gene editing has been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) and the membrane-associate accessory protein (MAAP) from an alternative open reading frame. AAP promotes capsid assembly, acting to target the capsid proteins to the nucleolus and promote capsid formation, MAAP enables cellular egress of fully formed particles. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV or rAAV) that contain a genome that lacks some, most or all of the native AAV genome and instead contains one or more heterologous sequences flanked by the ITRs have been successfully used in gene therapy and gene editing settings. These AAV vectors are widely used to deliver heterologous nucleic acid to cells of a subject. In some instances, the vectors are designed so that the heterologous nucleic acid integrates into the genome through homologous recombination (HR). One example of this is the ex vivo engineering of cells for immunotherapy, which is of increasing interest and AAV vectors have been used for the purpose of integrating heterologous nucleic acid into a host cell for use in immunotherapy. For example, AAV vectors have been used to produce chimeric antigen receptor (CAR) T cells, where the nucleic acid encoding the CAR is delivered to the T cell ex vivo and HR-mediated gene editing results in the CAR nucleic acid being integrated at a specific gene locus (see, e.g., Eyquem et al., 2017, Nature 543:113-119). However, the natural serotype AAV6, typically used to target primary T-cells, has not evolved to specifically drive such biomedical applications and is not necessarily the optimal AAV vector to mediate targeted gene editing in such cells. There remains a need, therefore, to a need to develop novel AAV vectors suitable for the delivery of nucleic acid to T cells.

### SUMMARY OF THE DISCLOSURE

The present disclosure is predicated in part on the identification of novel AAV capsid polypeptides. The capsid polypeptides, when present in the capsid of an AAV vector, can facilitate homology directed repair (HDR)-mediated gene editing of human T cells, typically at a level that is increased or enhanced compared to AAV vectors comprising a reference AAV capsid polypeptide (e.g., the prototypic AAV6 capsid set forth in SEQ ID NO:69). The capsid polypeptides of the present disclosure are therefore particularly useful in preparing AAV vectors, and in particular, AAV vectors for therapeutic applications. Similarly, AAV vectors comprising the capsid polypeptides of the present disclosure (i.e., having a capsid comprising or consisting of a capsid polypeptide of the present disclosure) are of particular use in delivering heterologous nucleic acids to T cells for use in immunotherapy, such as for the treatment of immunodeficiency.

In one aspect, provided is an AAV capsid polypeptide, comprising a peptide modification relative to the AAV6 capsid polypeptide set forth as SEQ ID NO:69, wherein the peptide modification comprises one or more or all of: a peptide insertion in variable region 8 (VR-VIII); 9 consecutive amino acids relative to the AAV6 polypeptide set forth in SEQ ID NO:69, comprising the sequence set forth in any one of SEQ ID NOs:70-97; and a substitution of one or more amino acids in the region of the capsid polypeptide spanning positions 1-170 with numbering relative to SEQ ID NO:69, wherein the capsid polypeptide comprises about 85%, 86%, 87%, 88%, 89%, 90%, 91%, 92%, 93%, 94%, 95%, 96%, 97%, 98% or 99% sequence identity to positions 1-170 of SEQ ID NO:69, and wherein the substituted amino acid sequence is derived from the AAV4 polypeptide set forth in SEQ ID NO:109.

In some examples, the peptide insertion in variable region 8 (VR-VIII) is in the region of the capsid polypeptide spanning positions 581-593, with numbering relative to SEQ ID NO:69. In one embodiment, the peptide modification comprises 9 consecutive amino acids, wherein 7 of those amino acids are insertions after position 588 relative to the AAV6 capsid polypeptide set forth in SEQ ID NO:69 and one amino acid substitution flanking either side of the insertion relative to the AAV6 capsid polypeptide set forth in SEQ ID NO:69. In further embodiments, the peptide modification comprises a substitution of one or more amino acids in the region of the capsid polypeptide spanning positions 19-162 with numbering relative to SEQ ID NO:69. In still further embodiments, the peptide modification comprises amino acid substitutions at positions 492, 705 and/or 731 relative to the AAV6 capsid polypeptide set forth in SEQ ID NO:69 (e.g., T492V, Y705F and/or Y731F, relative to the AAV6 capsid polypeptide set forth in SEQ ID NO:69). In some examples, the AAV capsid polypeptide comprises the sequence of amino acids set forth in any one of SEQ ID NOs:1-68 (e.g., SEQ ID NOs: 4, 49 and 59-61), or a sequence having at least or about 85%, 86%, 87%, 88%, 89%, 90%, 91%, 92%, 93%, 94%, 95%, 96%, 97%, 98%, or 99% sequence identity thereto.

In another aspect, provided is an AAV capsid polypeptide, comprising: a) a VP1 protein comprising the sequence of amino acids set forth in any one of SEQ ID NOs:1-68 (e.g., SEQ ID NO: 4, 49 and 59-61); b) a VP2 protein comprised within the sequence of amino acids set forth in any one of SEQ ID NOs:1-68 (e.g., SEQ ID NO: 4, 49 and 59-61); c) a VP3 protein comprised within the sequence of amino acids set forth in any one of SEQ ID NOs:1-68 (e.g., SEQ ID NO: 4, 49 and 59-61); or d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c).

Also provided are AAV vectors comprising a capsid polypeptide described above and herein. In some examples, the vector exhibits increased homology direct repair (HDR) efficiency of human T cells compared to an AAV vector comprising a capsid polypeptide comprising the sequence of amino acids set forth in SEQ ID NO:69. The AAV vector may further comprise a heterologous coding sequence, such as a heterologous coding sequence that encodes a peptide, polypeptide or polynucleotide (e.g., a therapeutic peptide, polypeptide or polynucleotide). In further embodiments, the AAV vector comprises a left homology arm and a right homology arm, wherein the sequence of the left homology arm and the sequence of the right homology arm are homologous to sequences at a locus in the genomic DNA of a host cell.

In further aspects, provided is an isolated nucleic acid molecule encoding a capsid polypeptide described herein, and a vector (e.g., a plasmid, cosmid, phage and transposon) comprising the nucleic acid molecule. Also provided is a host cell (e.g., a T cell), comprising an AAV vector, nucleic acid molecule or vector described herein.

In another aspect, provided is a method for introducing a heterologous coding sequence (e.g., a heterologous coding sequence encoding a chimeric antigen receptor) into a host cell, comprising contacting a host cell with an AAV vector of the disclosure that comprises a heterologous nucleic acid. In further embodiments, the host cell is also exposed to a genome editing nuclease (e.g., a zinc-finger nuclease (ZFN), transcription activator-like effector nucleases (TALEN) and clustered regularly interspaced short palindromic repeat (CRISPR)-Cas-associated nuclease) either before or after contacting the host cell with the AAV vector. In particular embodiments, the step of exposing the host cell to a genome editing nuclease comprises exposing the host cell to a ribonucleoprotein complex comprising a CRISPR-Cas-associated nuclease (e.g., Cas3, Cas9, Cas12 and Cas14) and a guide RNA (gRNA). In further embodiments, the host cell is a T cell (e.g., cytotoxic T cells, helper T cells, regulatory T cells, γδ T cells, αβ T cells and mucosal-associated invariant T (MAIT) cells). In particular examples, contacting a host cell with the AAV vector or the AAV vector and genome editing nuclease comprises administering the AAV vector or the AAV vector and genome editing nuclease to a subject. These methods can be in vivo, in vitro or ex vivo. In other examples, administration of the AAV vector to the subject effects treatment of an immunodeficiency.

Also provided is a method for producing an AAV vector, comprising culturing a host cell comprising a nucleic acid molecule encoding a capsid polypeptide described herein, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide, wherein the capsid encapsidates the heterologous coding sequence. In some examples, the host cell is a T cell.

In another aspect, provided is the use of an AAV vector described herein for the preparation of a medicament (e.g., a medicament for the treating an immunodeficiency).

### BRIEF DESCRIPTION OF THE DRAWINGS

Embodiments of the disclosure are described herein, by way of non-limiting example only, with reference to the following drawings.

FIG. 1 shows the detailed view of the modification workflow of the Ico6 capsid. The first step was the insertion of two SfiI restriction sites. The second step was the insertion of the peptide library. Seven truly randomized NNK or VNS (X1-X7) insertions as well as the full 9mer peptide including semi-random flanking amino acids are shown within the modified region. Amino acid position of Q585 and A592 using numbering from un-modified cap6 VP1. Abbreviations: lco: local codon-optimized.

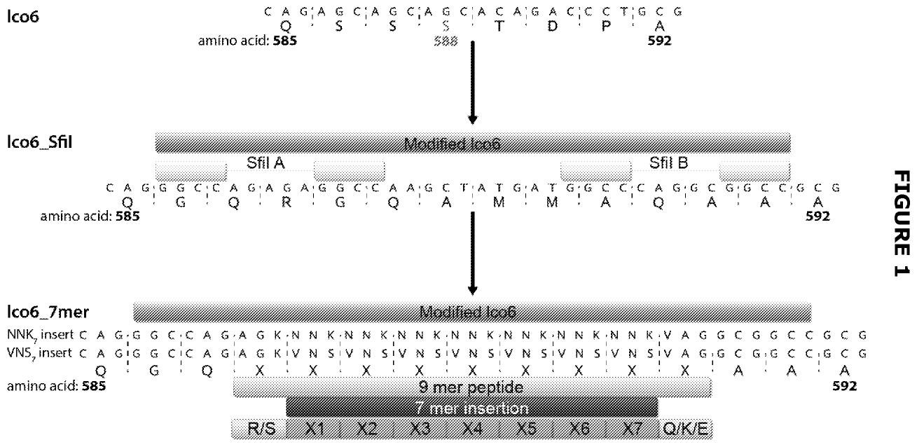

FIG. 2 shows the cross-over analysis on capsids recovered by subcloning and Sanger sequencing and by high-throughput PE300 NGS. Regions derived from AAV4 are depicted by * and regions derived from AAV6 are depicted by shading. The size bar and the depictions of the VP1, VP2, and VP3 proteins are a guide to help identify where the AAV4 contributions are located. The thick line above the size bar represents the 550 bp NGS amplicon for PE300 sequencing.

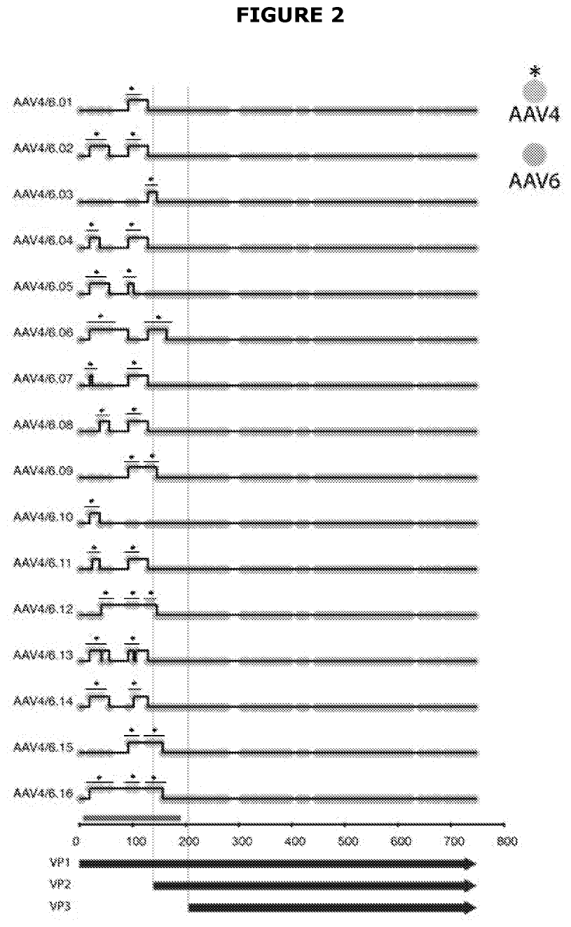

FIG. 3 shows (A) Length graph of the capsid proteins VP1, VP2, and VP3 including an indication which parts of the capsid harbor surface exposed residues. (B) Representative drawing of the location of the peptide insertions in parental capsid gene AAV6. (C) Representative drawing of the regions where chimeric AAV4 and AAV6 contributions were found in the selection of a shuffled library, all other parts of the capsid gene were purely derived from AAV6. (D) Representative drawing of the matured capsid genes harboring a chimeric 5′-end and a peptide insertion on the background of the AAV6 capsid gene. (E) Representative drawing of the matured capsid genes harboring a chimeric 5′-end, a peptide insertion, and previously published point mutation on the background of the AAV6 capsid gene. Amino acid residue numbering refers to the unmodified AAV6 capsid protein. All representative images also indicate the naming convention for all novel capsids based on their origin and modification, with X representing a number.

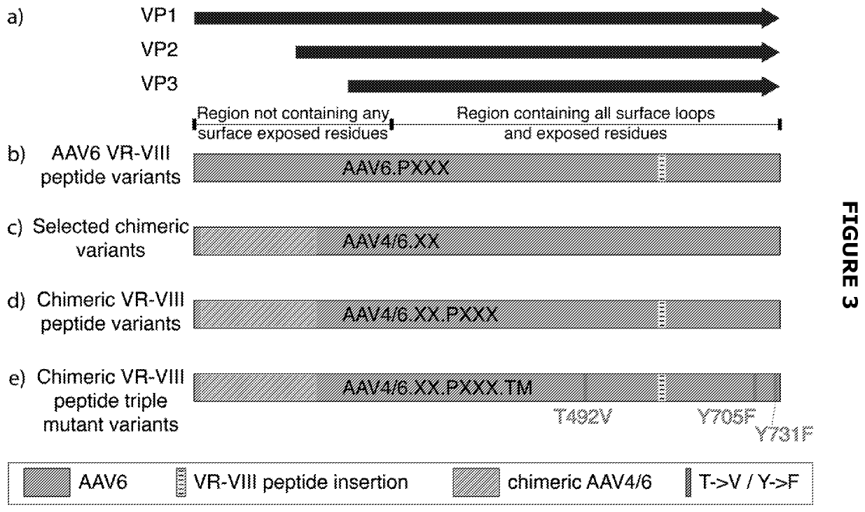

FIG. 4 shows the initial testing of novel AAV variants in human T cells. (A) A schematic representation of the study. T cells were electroporated with the Cas9/gRNA RNP complexes, followed by individual AAV transduction at a dose of 10,000 vg/cell. The AAVs packaged were CMV-driven barcoded GFP or promoter-less homology arm flanked barcoded GFP. Levels of HR (or homology directed repair; HDR) were evaluated by flow cytometry. (B) A graphical representation of homologous recombination efficiency (y-axis) and T cell expansion (x-axis) of novel AAV variants. AAV6 was used as the benchmark and all GFP expression was adjusted relative to AAV6.

FIG. 5 is a schematic representation of constructs used for validation of novel AAV capsids in T cells. (A) Conventional “AAV Kit” construct to gauge novel AAV capsids based on their efficiency to interact with cells (PCR amplified from whole cell DNA), enter the nucleus (PCR amplified from DNA in nuclear fraction), and express RNA (converted into cDNA and PCR amplified) in transduced cells. (B) Novel construct to investigate the performance of novel AAV capsids to mediate HDR by inserting a barcoded reporter into the TRAC locus and using nested PCR to amplify the barcode region. The same strategy was employed for hematopoietic stem and progenitor cells (HSPCs), with the homology arms substituted to match the BTK locus.

FIG. 6 shows the vector entry, nuclear translocation, and transgene expression of novel AAV capsids in T cells. (A) A schematic representation of the experimental workflow generating the data in FIG. 6B using the conventional “AAV Kit” construct described in FIG. 5A. (B) A heat map of novel AAV capsid performance at the total DNA, nuclear DNA, and transgene expression level. Medians from four different T-cell donors are shown, NGS read contributions were normalized to AAV6. Statistics: Kruskal-Wallis test 0.01*<0.05/0.001≤**<0.01/***<0.001.

FIG. 7 shows the homology-directed repair (HDR) gene editing performance of novel AAV capsids in T cells. (A) A schematic representation of the experimental workflow generating the data in FIG. 7B using the novel construct described in FIG. 5B. (B) A graphical representation of homologous recombination efficiency (y-axis) adjusted to the performance of AAV6. The x-axis shows the individual values and medians from four different T cell donors. NGS read contributions were normalized to AAV6. Statistics: Kruskal-Wallis test 0.05>*,≥0.01>**≥0.001>***>0.0001>****.

FIG. 8 shows enrichment of novel capsids over AAV6 in synthetic libraries selected for RNA expression and HDR efficiency in T cells. (A) A graphical representation of RNA expression (y-axis) of 200 capsids for each of the AAV2-derived and AAV6-derived libraries (x-axis). (B) A graphical representation of HDR performance (y-axis) of 200 capsids for each of the AAV2-derived and AAV6-derived libraries (x-axis). The best candidate from the RNA screen (AAV6.P20) and HDR screen (AAV6.P05) are indicated with arrows. Statistics: Kruskal-Wallis test ****<0.0001.

FIG. 9 shows protein expression from single-stranded transgenes in novel AAV capsids in T cells. (A) A schematic representation of the study. Primary human T cells from three donors were individually transduced with the novel AAV capsids and control AAV6. The AAVs packaged CMV-driven GFP flanked by TRAC HAs, enabling GFP expression following successful integration into the TRAC locus. Levels of HR (or homology directed repair; HDR) were evaluated 3 days after treatment by flow cytometry. (B) A graphical representation of homologous recombination efficiency (percentage of GFP-positive T cells; y-axis) of novel AAV variants (x-axis) normalized as fold change relative to AAV6. (C) A graphical representation of homologous recombination efficiency (mean fluorescence intensity, MFI; y-axis) of novel AAV variants (x-axis) normalized as fold change relative to AAV6.

FIG. 10 shows protein expression from self-complementary transgenes in novel AAV capsids in T cells. (A) A schematic representation of the study. Primary human T cells from three donors were individually transduced with the novel AAV capsids and control AAV6. The AAVs packaged CAG-driven GFP flanked by TRAC HAs, enabling GFP expression following successful integration into the TRAC locus. Levels of HR (or homology directed repair; HDR) were evaluated 3 days after treatment by flow cytometry. (B) A graphical representation of homologous recombination efficiency (percentage of GFP-positive T cells; y-axis) of novel AAV variants (x-axis) normalized as fold change relative to AAV6. (C) A graphical representation of homologous recombination efficiency (mean fluorescence intensity, MFI; y-axis) of novel AAV variants (x-axis) normalized as fold change relative to AAV6.

FIG. 11 shows HDR performance of novel AAV capsids in T cells. (A) A schematic representation of the study. Primary human T cells from nine (AAV6 and AAV6.P05) or six (all others) donors were individually transduced with the novel AAV capsids and control AAV6, following nucleofection of SpCas9/TRAC sgRNA RNPs, enabling GFP expression following successful integration into the TRAC locus. Levels of HR (or homology directed repair; HDR) were evaluated 5 days after treatment by flow cytometry. (B) A graphical representation of homologous recombination efficiency (y-axis) of novel AAV variants (x-axis) normalized as fold change to AAV6. Statistics: non-parametric Wilcoxon signed-rank test: p-value=ns≥0.05>*≥0.01>**≥0.001

FIG. 12 shows HDR performance of selected novel AAV capsids in T-cells. (A) Experimental workflow generating data in [B-D]. (B) Representative scatter plot showing the TCRαβ and GFP expression following RNP or mock electroporation and in absence or presence of the AAV4/6.15.P1. (C, D) Novel capsid performance at the HDR level. Shown are individual values and medians from six different T-cell donors. (C) Percent of GFP positive cells and (D) mean fluorescence intensities are shown. Statistics: Kruskal-Wallis test 0.05>*≥0.01>**≥0.001>***≥0.0001>****.

FIG. 13 shows the vector entry, nuclear translocation, and transgene expression of novel AAV capsids in HSPCs. (A) A schematic representation of the experimental workflow generating the data in FIG. 13B using the conventional “AAV Kit” construct described in FIG. 5A. (B) A heat map of novel AAV capsid performance at the total DNA, nuclear DNA, and transgene expression level. Medians from four different HSPC donors are shown. NGS read contributions were normalized to AAV6.

FIG. 14 shows HDR gene editing performance of novel AAV capsids in HSPCs. (A) A schematic representation of the experimental workflow generating the data in FIG. 14B using the novel construct described in FIG. 5B. (B) A graphical representation of homologous recombination efficiency (y-axis) adjusted to the performance of AAV6. The x-axis shows the medians from four different HSPC donors. NGS read contributions were normalized to AAV6.

FIG. 15 shows enrichment of novel capsids over AAV6 in synthetic libraries selected for RNA expression and HDR efficiency in HSPCs. (A) A graphical representation of RNA expression (y-axis) of 200 capsids for each of the AAV2-derived and AAV6-derived libraries (x-axis). (B) A graphical representation of HDR performance (y-axis) of 200 capsids for each of the AAV2-derived and AAV6-derived libraries (x-axis). The best candidate from the RNA screen (AAV6.P159), HDR screen (AAV6.P17) and the HDR screen in FIG. 12 (AAV6.P01) are indicated with arrows. Statistics: Mann-Whitney test ****<0.0001.

FIG. 16 shows the vector entry, nuclear translocation, and transgene expression of novel AAV capsids in murine immune cells. (A) A schematic representation of the experimental workflow generating the data in FIG. 16B using the conventional “AAV Kit” construct described in FIG. 5A. Heat maps of novel AAV capsid performance at the total DNA, nuclear DNA, and transgene expression level. Means from two different doses in murine (B) spleen derived activated T cells and (C) lineage negative bone marrow cells are shown. NGS read contributions were normalized to AAV6.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e., at least one or more than one) unless the context clearly dictates otherwise. Thus, for example, reference to “a polypeptide” includes a single polypeptide, as well as two or more polypeptides.

In the context of this specification, the term “about” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “host cell” refers to a cell, such as a mammalian cell, that has exogenous DNA introduced into it, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication-deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV6 is more accurately referred to as AAV6/6. A vector with an AAV6-derived capsid and an AAV4-derived genome is most accurately referred to as AAV4/6. A vector with the bioengineered DJ capsid and an AAV2-derived genome is most accurately referred to as AAV2/DJ. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett, 2001, Current Cardiology Reports, 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

The phrase “numbering relative to” a sequence, such as SEQ ID NO:69, means that the numbering of the amino acid position being referred to is as shown in the sequence, e.g. SEQ ID NO:69. It will be appreciated that the sequence is simply a reference sequence, and that the same amino acid residue or position may correspond to a different number in a different sequence, such as if the different sequence is a truncated form or is a sequence that has insertions or deletions compared to the reference sequence. To identify corresponding positions or residues in different sequences, sequences of related or variant polypeptides are aligned by any method known to those of skill in the art. Such methods typically maximize matches (e.g. identical nucleotides or amino acids at positions), and include methods such as using manual alignments and by using the numerous alignment programs available (for example, BLASTP, ClustlW, ClustlW2, EMBOSS, LALIGN, Kalign, etc.) and others known to those of skill in the art. By aligning the sequences of polypeptides, one skilled in the art can identify corresponding positions. For example, by aligning the prototypic AAV6 capsid polypeptide set forth in SEQ ID NO:69 with another AAV capsid polypeptide, such as the AAV6.P01 capsid set forth in SEQ ID NO:1, one of skill in the art can identify regions or amino acids residues within AAV6.P01 that correspond to various regions or residues in the AAV6 polypeptide set forth in SEQ ID NO:69.

As used herein, “corresponding nucleotides” or “corresponding amino acid residues” or grammatical variations thereof refer to nucleotides or amino acids that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc.) and others known to those of skill in the art. By aligning the sequences of polynucleotides or polypeptides, one skilled in the art can identify corresponding nucleotides or amino acids. For example, by aligning the prototypic AAV6 capsid polypeptide set forth in SEQ ID NO:69 with another AAV capsid polypeptide, such as the variant set forth in SEQ ID NO:1, one of skill in the art can identify regions or amino acids residues within the other AAV polypeptide that correspond to various regions or residues in the AAV polypeptide set forth in SEQ ID NO:69.

The term “peptide modification” refers to a modification in a polypeptide that involves two or more contiguous amino acids (i.e., that involves a peptide within the polypeptide). The peptide modification can include amino acid insertions, deletions and/or substitutions relative to a reference polypeptide. For example, an exemplary peptide modification of the present disclosure comprises 9 consecutive amino acid residues, wherein 7 of those residues are insertions relative to the prototypic AAV6 capsid set forth in SEQ ID NO:69, and 2 of those residues are amino acid substitutions relative to the prototypic AAV6 capsid set forth in SEQ ID NO:69.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “reporter gene” as used herein refers to a gene which encodes a gene product suitable for screening or sorting cells transduced with an AAV described herein that contains a genome comprising the reporter gene. The gene product can be any polypeptide or protein suitable for the intended use for screening technologies and can be cytoplasmic or membrane-bound. To facilitate sorting, the gene product can be directly detectable (e.g. may be a fluorescent protein), or may be indirectly-detectable, such as by using a labelled antibody that binds to the gene product. For the purposes of the present disclosure, the reporter gene does not encode an AAV capsid.

By “complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. Reference to “complementary” does not require complete or 100% complementarity, but can include less than complete or less than 100%, such as 70%, 75%, 80%, 85%, 90% or 95% complementarity. Standard Watson-Crick base-pairing includes: adenine/adenosine (A) pairing with thymidine/thymidine (T), A pairing with uracil/uridine (U), and guanine/guanosine (G) pairing with cytosine/cytidine (C). In addition, for hybridization between two RNA molecules (e.g., dsRNA), and for hybridization of a DNA molecule with an RNA molecule (e.g., when a target nucleic acid sequence base pairs with a gRNA) G can also base pair with U. For example, G/U base-pairing is partially responsible for the degeneracy (i.e., redundancy) of the genetic code in the context of tRNA anti-codon base pairing with codons in rnRNA. Thus, in the context of this disclosure, a G (e.g., of a target nucleic acid sequence base pairing with a gRNA) is considered complementary to both a U and to C. For example, when a G/U base-pair can be made at a given nucleotide position of a protein binding segment of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tₘ) for hybrids of nucleic acids having those sequences. Typically, the length for a hybridisable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

By “gene” it is meant a unit of inheritance that, when present in its endogenous state, occupies a specific locus on a genome and comprises transcriptional and/or translational regulatory sequences and/or a coding region and/or non-translated sequences (i.e., introns, 5′ and 3′ untranslated sequences).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “protein”, “peptide” and “polypeptide” are used interchangeably herein to refer to a polymer of amino acid residues linked together by peptide (amide) bonds. The terms refer to a protein, peptide, or polypeptide of any size, structure, or function.

As used herein, “genome editing” refers to the modification of the sequence of a host cell genome. The modification can include insertion and/or deletion of one or more nucleotides, and/or substitution or replacement of one or more nucleotides. Genome editing can be performed in vitro, in vivo or ex vivo.

The term “genome editing nuclease” refers to any enzyme that can catalyze the cleavage of phosphodiester bonds in nucleic acid, thereby facilitating or supporting genome editing.

The terms “guide RNA” or “gRNA” refer to a RNA sequence that is complementary to a target nucleic acid sequence and directs a RNA-guided nuclease to the target nucleic acid sequence. gRNA typically comprises CRISPR RNA (crRNA) and a tracr RNA (tracrRNA). “crRNA” is a 17-20 nucleotide sequence that is complementary to the target nucleic acid sequence, while the “tracrRNA” provides a binding scaffold for the RNA-guided nuclease. crRNA and tracrRNA exist in nature a two separate RNA molecules, which has been adapted for molecular biology techniques using, for example, 2-piece gRNAs such as CRISPR tracer RNAs (cr:tracrRNAs).

As used herein, a “homology arm” refers to a nucleic acid region or segment that has a sequence that is homologous to a genome on one or both sides of a target site in a genome locus, such that homologous recombination can occur between the genome and the homology arm, resulting in insertion of nucleic acid present between two homology arms at the target site, and/or removal of the equivalent nucleic acid from the native genome. The homology arms may have complete homology (i.e. 100% homology or sequence identity) or may have partial homology (e.g. 90%, 91%, 92%, 93%, 94%, 95%, 96%, 97%, 98% or 99% homology or sequence identity) to a sequence in the genome.

The terms “single-guide RNA” or “sgRNA” refer to a single RNA sequence that comprises the crRNA fused to the tracrRNA. Accordingly, the skilled person would understand that the term “gRNA” describes all CRISPR guide formats, including two separate RNA molecules or a single RNA molecule. By contrast, the term “sgRNA” will be understood to refer to single RNA molecules combining the crRNA and tracrRNA elements into a single nucleotide sequence.

The phrase “supports HDR-mediated gene editing” or grammatical variants thereof with respect to an AAV capsid polypeptide or AAV vector means that the AAV vector, or an AAV vector produced with the AAV capsid polypeptide can be used to deliver nucleic acid that can be incorporated into a host cell genome through a homologous recombination event. Integration of the nucleic acid into the genome through homologous recombination can be in the presence or absence of a genome editing nuclease. In some instances, the level or frequency of the HR-mediated gene editing that is supported by the AAV capsid polypeptide or the AAV vector is increased compared to a reference AAV vector or AAV capsid, such as by 20%, 30%, 40%, 50%, 60%, 70%, 80%, 90%, 100%, 150%, 200%, 250%, 300% or more.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

It will be appreciated that the above described terms and associated definitions are used for the purpose of explanation only and are not intended to be limiting.

TABLE 1
Description of the Sequences

| SEQ | |
|---|---|
| ID NO. | Sequence description |
| 1 | AAV6.P01 (VP1 protein) |
| 2 | AAV6.P02 (VP1 protein) |
| 3 | AAV6.P04 (VP1 protein) |
| 4 | AAV6.P05 (VP1 protein) |
| 5 | AAV6.P08 (VP1 protein) |
| 6 | AAV6.P09 (VP1 protein) |
| 7 | AAV6.P013 (VP1 protein) |
| 8 | AAV6.P017 (VP1 protein) |
| 9 | AAV6.P020 (VP1 protein) |
| 10 | AAV6.P027 (VP1 protein) |
| 11 | AAV6.P046 (VP1 protein) |
| 12 | AAV6.P049 (VP1 protein) |
| 13 | AAV6.P051 (VP1 protein) |
| 14 | AAV6.P057 (VP1 protein) |
| 15 | AAV6.P059 (VP1 protein) |
| 16 | AAV6.P061 (VP1 protein) |
| 17 | AAV6.P065 (VP1 protein) |
| 18 | AAV6.P072 (VP1 protein) |
| 19 | AAV6.P081 (VP1 protein) |
| 20 | AAV6.P084 (VP1 protein) |
| 21 | AAV6.P090 (VP1 protein) |
| 22 | AAV6.P093 (VP1 protein) |
| 23 | AAV6.P098 (VP1 protein) |
| 24 | AAV6.P099 (VP1 protein) |
| 25 | AAV6.P108 (VP1 protein) |
| 26 | AAV6.P123 (VP1 protein) |
| 27 | AAV6.P147 (VP1 protein) |
| 28 | AAV6.P187 (VP1 protein) |
| 29 | AAV4/6.01 (VP1 protein) |
| 30 | AAV4/6.02 (VP1 protein) |
| 31 | AAV4/6.03 (VP1 protein) |
| 32 | AAV4/6.04 (VP1 protein) |
| 33 | AAV4/6.05 (VP1 protein) |
| 34 | AAV4/6.06 (VP1 protein) |
| 35 | AAV4/6.07 (VP1 protein) |
| 36 | AAV4/6.08 (VP1 protein) |
| 37 | AAV4/6.09 (VP1 protein) |
| 38 | AAV4/6.10 (VP1 protein) |
| 39 | AAV4/6.11 (VP1 protein) |
| 40 | AAV4/6.12 (VP1 protein) |
| 41 | AAV4/6.13 (VP1 protein) |
| 42 | AAV4/6.14 (VP1 protein) |
| 43 | AAV4/6.15 (VP1 protein) |
| 44 | AAV4/6.16 (VP1 protein) |
| 45 | AAV4/6.02.P01 (VP1 protein) |
| 46 | AAV4/6.02.P02 (VP1 protein) |
| 47 | AAV4/6.02.P04 (VP1 protein) |
| 48 | AAV4/6.02.P06 (VP1 protein) |
| 49 | AAV4/6.02.P13 (VP1 protein) |
| 50 | AAV4/6.02.P17 (VP1 protein) |
| 51 | AAV4/6.11.P01 (VP1 protein) |
| 52 | AAV4/6.11.P02 (VP1 protein) |
| 53 | AAV4/6.11.P03 (VP1 protein) |
| 54 | AAV4/6.11.P04 (VP1 protein) |
| 55 | AAV4/6.11.P06 (VP1 protein) |
| 56 | AAV4/6.11.P13 (VP1 protein) |
| 57 | AAV4/6.11.P17 (VP1 protein) |
| 58 | AAV4/6.15.P01 (VP1 protein) |
| 59 | AAV4/6.15.P05 (VP1 protein) |
| 60 | AAV4/6.16.P01 (VP1 protein) |
| 61 | AAV4/6.16.P05 (VP1 protein) |
| 62 | AAV4/6.02.P01.TM (VP1 protein) |
| 63 | AAV4/6.02.P04.TM (VP1 protein) |
| 64 | AAV4/6.02.P13.TM (VP1 protein) |
| 65 | AAV4/6.11.P01.TM (VP1 protein) |
| 66 | AAV4/6.15.P01.TM (VP1 protein) |
| 67 | AAV4/6.15.P05.TM (VP1 protein) |
| 68 | AAV4/6.16.P05.TM (VP1 protein) |
| 69 | AAV6 prototypic capsid (VP1 protein), GenBank AAB95450.1 |
| 70 | RSEKELEEK peptide insert |
| 71 | SEEGETGKK peptide insert |
| 72 | SNNISDKDQ peptide insert |
| 73 | SADDVTKDK peptide insert |
| 74 | SDDDNTKTK peptide insert |
| 75 | SDDDNSKIK peptide insert |
| 76 | SDENNMKDK peptide insert |
| 77 | SEDHADKDK peptide insert |
| 78 | SDANEKKEQ peptide insert |
| 79 | SEEVGGKDK peptide insert |
| 80 | SEAVEGKEK peptide insert |
| 81 | SNESEKKEK peptide insert |
| 82 | SIETEPKEK peptide insert |
| 83 | SVETEGKEK peptide insert |
| 84 | SEGDAVDKK peptide insert |
| 85 | SEMEAKPEK peptide insert |
| 86 | STNDDSKEK peptide insert |
| 87 | STADDDKTK peptide insert |
| 88 | SGKPEEKEK peptide insert |
| 89 | SSEENTKEK peptide insert |
| 90 | SDTADHKEK peptide insert |
| 91 | SGDGDIKEK peptide insert |
| 92 | SQTEGEKEK peptide insert |
| 93 | SVEGGEKEK peptide insert |
| 94 | SDVQGEKEK peptide insert |
| 95 | SEDMGSKEK peptide insert |
| 96 | SDDQGSKEK peptide insert |
| 97 | SDLDPDKIK peptide insert |
| 98 | eGFP forward primer |
| 99 | eGFP reverse primer |
| 100 | TRAC genomic forward primer |
| 101 | BTK genomic forward primer |
| 102 | AAV2/AAV6 peptide forward primer |
| 103 | AAV2/AAV6 peptide reverse primer |
| 104 | AAV4/AAV6 shuffled forward primer |
| 105 | AAV4/AAV6 shuffled reverse primer 1 |
| 106 | AAV4/AAV6 shuffled reverse primer 2 |
| 107 | TRAC gRNA |
| 108 | BTK gRNA |
| 109 | AAV4 prototypic capsid (VP1 protein), GenBank NP_044927.1 |

### Capsid Polypeptides

The present disclosure is predicated, at least in part, on the identification of novel AAV capsid polypeptides. The capsid polypeptides, when present in the capsid of an AAV vector, can facilitate homology directed repair (HDR)-mediated gene editing of cells, and in particular HDR-mediated gene editing of T cells (e.g. human T cells). The HDR efficiency of T cells by AAV vectors having a capsid comprising a capsid polypeptide of the present disclosure is generally increased or enhanced compared to AAV vectors comprising a reference AAV capsid polypeptide (e.g. the prototypic AAV6 capsid set forth in SEQ ID NO:69). The level or frequency of the HR-mediated gene editing that is supported by the AAV capsid polypeptide or the AAV vector described herein is increased compared to a reference AAV vector or AAV capsid, such as by 20%, 30%, 40%, 50%, 60%, 70%, 80%, 90%, 100%, 150%, 200%, 250%, 300% or more.

The capsid polypeptides of the present disclosure are therefore particularly useful in preparing AAV vectors, and in particular AAV vectors for delivery of heterologous nucleic acid to T cells, for use in immunotherapy, e.g., CAR-T therapy. In exemplary embodiments, the capsid polypeptides of the present disclosure are useful in preparing AAV vectors for treating immunodeficiency.

Thus, in one aspect, provided is an AAV capsid polypeptide, comprising a peptide modification relative to the AAV6 polypeptide set forth in SEQ ID NO:69, wherein the peptide modification comprises one or more or all of:
a) a peptide insertion in variable region 8 (VR-VIII);
b) 9 consecutive amino acids relative to the AAV6 polypeptide set forth in SEQ ID NO:69, comprising the sequence set forth in any one of SEQ ID NOs:70-97; and
c) a substitution of one or more amino acids in the region of the capsid polypeptide spanning positions 1-170 with numbering relative to SEQ ID NO:69, wherein the capsid polypeptide comprises about 85%, 86%, 87%, 88%, 89%, 90%, 91%, 92%, 93%, 94%, 95%, 96%, 97%, 98% or 99% sequence identity to positions 1-170 of SEQ ID NO:69 and wherein the substituted amino acid sequence is derived from the AAV4 polypeptide set forth in SEQ ID NO:109.

The AAV capsid polypeptides of the present disclosure include those having a peptide modification in variable region 8 (VR-VIII) relative to a reference AAV capsid polynucleotide, such as the prototypic AAV6 capsid polypeptide set forth in SEQ ID NO:69 (where VR-VIII spans amino acids 581-593 of SEQ ID NO:69). In some embodiments, the peptide modification comprises 9 consecutive amino acids, wherein 7 of those amino acids are insertions after position 588 relative to the AAV6 capsid polypeptide set forth in SEQ ID NO:69. In another embodiment, the peptide modification comprises 9 consecutive amino acids, wherein 7 of those amino acids are insertions after position 588 and one amino acid substitution flanking either side of the insertion relative to the AAV6 capsid polypeptide set forth in SEQ ID NO:69. Typically, the peptide modification comprises the 9 consecutive amino acid residues having a sequence set forth in any one of SEQ ID NOs:70-97. The peptide modification can be at any location in the VR-VIII.

In an embodiment, the peptide modification comprises a substitution of one or more amino acids in the region of the capsid polypeptide spanning positions 15-165 with numbering relative to SEQ ID NO:69, and wherein the substituted amino acid sequence is derived from the AAV4 polypeptide set forth in SEQ ID NO:109. In another embodiment, the peptide modification comprises amino acid substitutions at positions 492, 705 and/or 731 relative to the AAV6 capsid polypeptide set forth in SEQ ID NO:6 (e.g., T492V, Y705F and/or Y731F, relative to the AAV6 capsid polypeptide set forth in SEQ ID NO:69, as described by Ling et al., 2016, Scientific Reports, 6: 35495). In some examples, the AAV capsid polypeptide comprises the sequence of amino acids set forth in any one of SEQ ID NOs:1-68 (e.g., SEQ ID NOs: 4, 49 and 59-61), or a sequence having at least or about 85%, 86%, 87%, 88%, 89%, 90%, 91%, 92%, 93%, 94%, 95%, 96%, 97%, 98%, or 99% sequence identity thereto. The AAV capsid polypeptides of the present disclosure can include all or a portion of the VP1 protein, VP2 protein and/or the VP3 protein. The AAV capsid polypeptides typically comprise at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the VP1, VP2 or VP3 proteins of the prototypic AAV6 set forth in SEQ ID NO:69.

Thus, provided herein are polypeptides, including isolated polypeptides, comprising all or a portion of an AAV capsid polypeptide set forth in any one of SEQ ID NOs:1-68, or a polypeptide comprising at least or about 85%, 86%, 87%, 88%, 89%, 90%, 91%, 92%, 93%, 94%, 95%, 96%, 97%, 98%, or 99% sequence identity thereto. Also included in the present disclosure are AAV capsid polypeptides comprising all or a portion of the VP2 protein set comprised in any one of SEQ ID NOs:1-68 or comprising a sequence having at least or about 85%, 86%, 87%, 88%, 89%, 90%, 91%, 92%, 93%, 94%, 95%, 96%, 97%, 98%, or 99% sequence identity to the VP2 protein comprised in any one of SEQ ID NOs:1-68 or a functional fragment thereof. In addition, provided are AAV capsid polypeptides comprising all or a portion of the VP3 protein comprised in any one of SEQ ID NOs:1-68 or comprising a sequence having at least or about 85%, 86%, 87%, 88%, 89%, 90%, 91%, 92%, 93%, 94%, 95%, 96%, 97%, 98%, or 99% sequence identity to the VP3 protein comprised in any one of SEQ ID NOs:1-68 or a functional fragment thereof.

In another aspect, provided is an AAV capsid polypeptide, comprising: a) a VP1 protein comprising the sequence of amino acids set forth in any one of SEQ ID NOs:1-68 (e.g., SEQ ID NO: 4, 49 and 59-61); b) a VP2 protein comprised within the sequence of amino acids set forth in any one of SEQ ID NOs:1-68 (e.g., SEQ ID NO: 4, 49 and 59-61); c) a VP3 protein comprised within the sequence of amino acids set forth in any one of SEQ ID NOs:1-68 (e.g., SEQ ID NO: 4, 49 and 59-61); or d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c).

An exemplary capsid polypeptide, AAV6.P05 (SEQ ID NO:4) comprises a peptide modification in VR-VIII relative to SEQ ID NO:69, wherein the peptide modification comprises the sequence SEAVEGKEK (SEQ ID NO:80), which includes a 7 amino acid insertion after position 588 relative to the prototypic AAV6 capsid polypeptide set forth in SEQ ID NO:69 and one amino acid substitution flanking either side of the insertion relative to the AAV6 capsid polypeptide set forth in SEQ ID NO:69. Thus, in some embodiments, provided are capsid polypeptides comprising a peptide modification in VR-VIII relative to SEQ ID NO:69, wherein the peptide modification comprises the sequence SEAVEGKEK (SEQ ID NO:80). Thus, in some embodiments, provided are AAV capsid polypeptides comprising a peptide modification in VR-VIII relative to SEQ ID NO:69, wherein the peptide modification comprises the sequence SEAVEGKEK (SEQ ID NO:80), and wherein the capsid polypeptide has at least or about 95%, 96%, 97%, 98%, or 99% sequence identity to the VP1 protein set forth in SEQ ID NO:69.

Another exemplary capsid polypeptide, AAV4/6.02.P13 (SEQ ID NO:49) comprises a peptide modification in VR-VIII relative to SEQ ID NO:69, wherein the peptide modification comprises the sequence SNNISDKDQ (SEQ ID NO:72), which includes a 7 amino acid insertion after position 588 relative to the prototypic AAV6 capsid polypeptide set forth in SEQ ID NO:69 and one amino acid substitution flanking either side of the insertion relative to the AAV6 capsid polypeptide set forth in SEQ ID NO:69. Thus, in some embodiments, provided are capsid polypeptides comprising a peptide modification in VR-VIII relative to SEQ ID NO:69, wherein the peptide modification comprises the sequence SNNISDKDQ (SEQ ID NO:72), and wherein the capsid polypeptide has at least or about 95%, 96%, 97%, 98%, or 99% sequence identity to the VP1 protein set forth in SEQ ID NO:69. In particular embodiments, the peptide modification comprises a substitution of one or more amino acids in the region of the capsid polypeptide spanning positions 1-170 with numbering relative to SEQ ID NO:69, wherein the substituted amino acid sequence is derived from the AAV4 polypeptide set forth in SEQ ID NO:109, and wherein the capsid polypeptide comprises about 90%, 91%, 92%, 93%, 94%, 95% or 96% sequence identity to positions 1-170 of SEQ ID NO:69.

A further exemplary capsid polypeptide, AAV4/6.15.P05 (SEQ ID NO:59) comprises a peptide modification in VR-VIII relative to SEQ ID NO:69, wherein the peptide modification comprises the 9 consecutive amino acids SEAVEGKEK (SEQ ID NO:80), which includes a 7 amino acid insertion after position 588 relative to the prototypic AAV6 capsid polypeptide set forth in SEQ ID NO:69, and one amino acid substitution flanking either side of the insertion relative to the AAV6 capsid polypeptide set forth in SEQ ID NO:69. Thus, in some embodiments, provided are AAV capsid polypeptides comprising a peptide modification in VR-VIII relative to SEQ ID NO:69, wherein the peptide modification comprises the 9 consecutive amino acids SEAVEGKEK (SEQ ID NO:80), and wherein the capsid polypeptide has at least or about 90%, 91%, 92%, 93%, 94%, 95%, 96%, 97%, 98%, or 99% sequence identity to the VP1 protein set forth in SEQ ID NO:69. In particular embodiments, the peptide modification comprises a substitution of one or more amino acids in the region of the capsid polypeptide spanning positions 1-170 with numbering relative to SEQ ID NO:69, wherein the substituted amino acid sequence is derived from the AAV4 polypeptide set forth in SEQ ID NO:109, and wherein the capsid polypeptide comprises about 89%, 90%, 91%, 92%, 93%, 94% or 95% sequence identity to positions 1-170 of SEQ ID NO:69.

A further exemplary capsid polypeptide, AAV4/6.16.P01 (SEQ ID NO:60) comprises a peptide modification in VR-VIII relative to SEQ ID NO:69, wherein the peptide modification comprises the 9 consecutive amino acids SEEVGGKDK (SEQ ID NO:79), which includes a 7 amino acid insertion after position 588 relative to the prototypic AAV6 capsid polypeptide set forth in SEQ ID NO:69 and one amino acid substitution flanking either side of the insertion relative to the AAV6 capsid polypeptide set forth in SEQ ID NO:69. Thus, in some embodiments, provided are AAV capsid polypeptides comprising a peptide modification in VR-VIII relative to SEQ ID NO:69, wherein the peptide modification comprises the 9 consecutive amino acids SEEVGGKDK (SEQ ID NO:79), and wherein the AAV capsid polypeptide has at least or about 90%, 91%, 92%, 93%, 94%, 95%, 96%, 97%, 98%, or 99% sequence identity to the VP1 protein set forth in SEQ ID NO:69. In particular embodiments, the peptide modification comprises a substitution of one or more amino acids in the region of the capsid polypeptide spanning positions 1-170 with numbering relative to SEQ ID NO:69, wherein the substituted amino acid sequence is derived from the AAV4 polypeptide set forth in SEQ ID NO:109, and wherein the capsid polypeptide comprises about about 85%, 86%, 87%, 88%, 89% or 90% sequence identity to positions 1-170 of SEQ ID NO:69.

Another exemplary capsid polypeptide, AAV4/6.16.P05 (SEQ ID NO:61) comprises a peptide modification in VR-VIII relative to SEQ ID NO:69, wherein the peptide modification comprises the 9 consecutive amino acids SEAVEGKEK (SEQ ID NO:80), which includes a 7 amino acid insertion after position 588 relative to the prototypic AAV6 capsid polypeptide set forth in SEQ ID NO:69 and one amino acid substitution flanking either side of the insertion relative to the AAV6 capsid polypeptide set forth in SEQ ID NO:69. Thus, in some embodiments, provided are capsid polypeptides comprising a peptide modification in VR-VIII relative to SEQ ID NO:69, wherein the peptide modification comprises the 9 consecutive amino acids SEAVEGKEK (SEQ ID NO:80), and wherein the capsid polypeptide has at least or about 90%, 91%, 92%, 93%, 94%, 95%, 96%, 97%, 98%, or 99% sequence identity to the VP1 protein set forth in SEQ ID NO:69. In particular embodiments, the peptide modification comprises a substitution of one or more amino acids in the region of the capsid polypeptide spanning positions 1-170 with numbering relative to SEQ ID NO:69, wherein the substituted amino acid sequence is derived from the AAV4 polypeptide set forth in SEQ ID NO:109, and wherein the capsid polypeptide comprises about about 85%, 86%, 87%, 88%, 89% or 90% sequence identity to positions 1-170 of SEQ ID NO:69.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell). Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein. Methods for vectorizing a capsid protein are well known in the art and any suitable method can be employed for the purposes of the present disclosure. For example, the cap gene can be recovered (e.g., by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, HeLa cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g., miRNA, siRNA, and shRNA). As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e., gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instance, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject, treats an immunodeficiency (i.e., a primary immunodeficiency disease or disorder). In illustrative embodiments, the immunodeficiency is selected from among a B cell deficiencies (including common variable immunodeficiency, selective IgA deficiency, Brunton's or X-linked agammaglobulinemia), T cell deficiencies (including severe combined immunodeficiency, DiGeorge syndrome, Wiskott-Aldrich syndrome, Ataxia-telangiectasia, X-linked hyper IgM), combination B and T cell deficiencies, defective phagocytic disorders (including chronic granulomatous disease), complement disorders, and idiopathic diseases or disorders (i.e., of unknown origin). Those skilled in the art would readily be able to select an appropriate heterologous coding sequence useful for treating such diseases. In some examples, the heterologous coding sequence comprises all or a part of a gene that is associated with the disease, such as all or a part of a gene set forth in Table 2. Introduction of such a sequence to the immune cells can be used for gene replacement or gene editing/correction, e.g. using CRISPR-Cas9. In particular examples, the heterologous coding sequence encodes a protein encoded by a gene that is associated with the disease, such as a gene set forth in Table 2.

TABLE 2

| Exemplary diseases or | |
|---|---|
| disorders | Exemplary associated genes |
| Combined | WAS, ATM, 22Q11.2DS, STAT3, STAT5B, |
| immunodeficiencies | DNMT3B, ZBTB2, RMRP, TTC7A, HOIP, |
| | SP11, MYSM1 |
| T cell deficiencies | TFRC, DOCK2, MYSM1, NEIL1 |
| B cell deficiencies | BTK, AICDA, NFKB2 |
| Defective phagocytic | CYBA, NCF1, NCF2, JAGN1, CSF2RB |
| disorders | |

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion, or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

In an embodiment, the vector further comprises a left homology arm and a right homology arm, wherein the sequence of the left homology arm and the sequence of right homology arm are homologous to sequences at a locus in the genomic DNA of the host cell.

Each of the homology arms (i.e., the left or 3′ homology arm, and the right or 5′ homology arm) have a sequence that is homologous to a sequence in the genomic DNA at the locus being targeted for HR-mediated gene editing. The left or 3′ homology arm is homologous to a region that is upstream of (or 3′ to) the targeted site of integration, while the right or 5′ homology arm is homologous to a region that is downstream of (or 5′ to) the targeted site of integration. The homology arms are therefore used to target the locus and facilitate or enable HDR. For the purposes of the present disclosure, the homology arms are typically about 50 bp to about 800 bp, about 150 bp to about 750 bp, or about 300 bp to about 700 bp in length (e.g., so as to keep within the permitted or optimal genome length of the AAV genome).

The homology arms can be designed to be homologous to, and thus target the heterologous coding sequence to, any desired locus in the genomic DNA of the host cell. In one example, the locus is the T-cell receptor a constant (TRAC) locus and the homology arms are homologous to regions in the TRAC gene. As described in Eyquem et al. (2017, supra), targeting a chimeric antigen receptor (CAR) to this locus results in uniform expression of the CAR in human peripheral blood T cells and enhances T cell potency. Thus, selecting and identifying AAV variants that effectively support HR-mediated integration of a nucleic acid at the TRAC locus of a T cell would be of benefit for the ex vivo production of CAR T cells. Other loci that are particularly relevant to T cells include the T cell receptor β-chain constant region 1 (TRBC1), human leukocyte antigen (HLA), CD52, PD-1, IL2Ra, B2M, CD7, and CTLA-4 (for review, see Atsavapranee et al., 2021, EBioMedicine, 67:103354).

In some examples, the AAV comprises a promoter that is functional in the host cell. In some examples, the promoter is constitutive in the host cells used for selection and potentially also for the downstream therapeutic applications. In particular examples, the promoter is a ubiquitous promoter (i.e., functional in multiple tissue types or multiple host cells) and/or a constitutive promoter. In other examples, the promoter is tissue-specific. Suitable promoters are well known to those skilled in the art and non-limiting examples include AAV promoters (e.g., the p5, p19 or p40 promoters), constitutive promoters (e.g., the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EFla promoter), inducible promoters (e.g., the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system), inducible promoters which may be useful in this context are those which are regulated by a specific physiological state, e.g., temperature, acute phase, a particular differentiation state of the cell, or in replicating cells only (e.g., the liver-specific thyroxin binding globulin (TBG) promoter, insulin promoter, glucagon promoter, somatostatin promoter, pancreatic polypeptide (PPY) promoter, synapsin-1 (Syn) promoter, creatine kinase (MCK) promoter, mammalian desmin (DES) promoter, a α-myosin heavy chain (a-MHC) promoter, a cardiac Troponin T (cTnT) promoter, beta-actin promoter, and hepatitis B virus core promoter). The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element (RTE), and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for human growth hormone (HGH) and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a AAV capsid polypeptide, AAV vector, nucleic acid molecule, or vector of the present disclosure. In some instances, the host cells are used to amplify, replicate, package and/or purify a polynucleotide or vector. In other examples, the host cells are used to express a heterologous coding sequence, such as one packaged within the AAV vector. Exemplary host cells include prokaryotic and eukaryotic cells. In some instances, the host cell is a mammalian host cell. It is well within the skill of a skilled artisan to select an appropriate host cell for the expression, amplification, replication, packaging and/or purification of a polynucleotide, vector or rAAV virion of the present disclosure. Exemplary mammalian host cells include, but are not limited to, HEK293 cells, HeLa cells, Vero cells, HuH-7 cells, and HepG2 cells. Exemplary host cells are T cells (including αβ T cells, cytotoxic T cells, helper T cells, regulatory T cells, γδ T cells, and mucosal-associated invariant T (MAIT) cells).

### Compositions and Methods

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The AAV vectors of the present disclosure, and compositions containing the AAV vectors, may be used in methods for the introduction of a heterologous coding sequence into a host cell. Such methods involve contacting the host cell with the AAV vector (or the AAV vector and genomic editing nuclease). This may be performed in vitro, ex vivo or in vivo. In particular embodiments, the host cell is a T cell, such as a primary human T cell.

When the methods are performed ex vivo or in vivo, typically the introduction of the heterologous sequence into the host cell is for therapeutic purposes, whereby expression of the heterologous sequence results in the treatment of a disease or condition (e.g., an immunodeficiency). Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intravenous, intraperitoneal, subcutaneous, epicutaneous, intradermal, intramuscular, pulmonary, intracranial, intraosseous, oral, buccal, or nasal routes. The AAV vector can be administered as a single dose or multiple doses, and at varying intervals.

Also provided are methods for introducing a heterologous coding sequence into a host cell. Such methods comprise contacting a host cell with the AAV vector described herein.

In some embodiments, the methods further comprise exposing to host cell to a genome editing nuclease. Exposure of the host cells to a genome editing nuclease, either before or after the cells are transduced with the AAV vector, enhances HDR by inducing double-stranded breaks (DSBs) in the genomic DNA at the locus. These DSBs are then repaired by homology directed repair (HDR) using the templates of the homology arms, resulting in integration of the heterologous coding sequence at the locus.

Any suitable genome editing nuclease can be used, including CRISPR-associated protein (Cas) endonucleases, zinc finger nucleases (ZFNs), transcription activator-like effector nucleases (TALENs), meganucleases, variants, fragments and combinations thereof. Naturally-occurring and synthetic genome editing nuclease are contemplated herein.

In one embodiment, the genome editing system is a CRISPR-Cas genome editing system. The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546). Briefly, upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence. Suitable Cas endonucleases for the methods of the present disclosure would be known to persons skilled in the art, illustrative examples of which include Cas3, Cas9, Cas12 (e.g., Cas12a, Cas12b, Cas12c, Cas12d, Cas12e) and Cas14.

Thus, in one example, the host cells are exposed to a ribonucleoprotein (RNP) complex comprising a Cas endonuclease and suitable guide RNA (gRNA) specific for the loci. Methods and tools for the design of gRNA would be known to persons skilled in the art, illustrative examples of which include CHOPCHOP, CRISPR Design, sgRNA Designer, Synthego and GT-Scan. Suitable gRNAs would be known to persons skilled in the art or could be designed and produced by persons skilled in the art, illustrative examples of which include the gRNAs described elsewhere and herein, such as the gRNA targeting TRAC set forth in SEQ ID NO: 108 or the gRNA described in in Eyquem et al. (2017, supra).

Exposure of the cells to the genome editing nuclease, including the RNP complex, can be by any suitable means, such as electroporation, nucleofection, and lipid-mediated transfection.

In some embodiments, the host cell is a T cell. Where the host cell is a T cell, it is also contemplated herein that the AAV vectors may be used for the generation/production of chimeric antigen receptor (CAR) T cells. As such, in some embodiments, the heterologous coding sequence encodes a CAR.

Also provided are methods for producing an AAV vector described above and herein, i.e., one comprising a AAV capsid polypeptide of the present disclosure. Such methods comprise culturing a host cell comprising a nucleic acid molecule encoding an AAV capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### Examples

### Example 1. Materials and Methods

### Cell Culture Conditions and Origins

AAV production and transduction experiments were performed in a validated human embryonic kidney HEK293T cell line and grown in Dulbecco's modified Eagle's medium (DMEM) supplemented with 10% fetal bovine serum, 1× Pen Strep and 25 mM HEPES. Primary human T cells derived from peripheral blood mononuclear cells of adult healthy volunteers using Lymphoprep (StemCell Technologies) and isolated by EasySep Release Human CD3-positive selection kit (StemCell Technologies). T cells were activated using CD3/CD28 T Cell TransAct microbeads (Mytenyi Biotec) and expanded using OpTmizer T Cell Expansion SFM (Gibco) supplemented with Immune Cell SR (Gibco), 10 ng/mL hIL-7 and 50 IU/mL hIL-2.

Selection of Novel AAV Capsids in T Cells Novel AAV capsids were selected in T cells using a two-round selection process described by Westhaus et al. (2022, Human Gene Therapy, in press, DOI: 10.1089/hum.2021.278) for optimal RNA expression and a homologous recombination (HR) selection platform for optimal HDR. The AAV capsid libraries used in this study were an AAV2 and AAV6 VR-VIII peptide insertion capsid libraries and an AAV4/AAV6 shuffled capsid library.

### AAV Production

The novel AAV peptide insertion and shuffled variants were produced in vitro in 5×15 cm dishes of HEK293T cells to package ITR2-CMVp-eGFP-N₆Barcode(BC)-WPRE-ITR2 transgenes as described previously (Westhaus et al., 2020, Human Gene Therapy, 31(9 and 10): 575-589; Cabanes-Creus et al., 2020(A), Science Translational Medicine, 12(560): eaba3312; Cabanes-Creus et al., 2020(B), Molecular Therapy: Methods & Clinical Development, 17: 1139-1154). Selected constructs were harvested and purified using iodixanol ultra-centrifugation, as described previously (Cabanes-Creus et al., 2020(B), supra).

Iodixanol-produced AAV were titrated using real-time quantitative polymerase chain reaction (qPCR) master mix (Cat #172-5125; Bio-Rad) with serial dilutions of a linearized plasmid as a standard curve and eGFP primers (F: 5′-TCAAGATCCGCCACAACATC-3′, SEQ ID NO:98; R: 5′-TTCTCGTTGGGGTCTTTGCT-3′, SEQ ID NO:99) as previously described (Cabanes-Creus et al., 2020(B), supra).

The resulting AAVs were titrated using qPCR was performed using standard protocols. Dilutions of 1/10 and 1/100 were used for media, and dilutions of 1/100 and 1/1000 were used for cell lysates. Primers used included eGFP_F/R (SEQ ID NO:98 and 99). Cycle: 98° C. 2 min, 39 times 98° C. 5s+60° C. 15s, 65° C. 30s, melting curve from 65° C. to 95° C. by adding 0.5° C. each 5s. Titers were averaged on the 6 measures done for each sample (2 dilutions×3 replicates) and the lysate titer was added to the media titer to obtain total titer.

### RNA Extraction and Reverse Transcription (RT)

RNA was extracted from pelleted cells following QIAGEN's DNA/RNA AllPrep protocol. To further purify RNA, 0.5 μg of RNA were incubated 3 h at 37° C. with 1 μL of ThermoFisher's TURBO DNase. DNase was inactivated using beads provided. Oligo dT primers and dNTP (to a final concentration of 1 mM) were added to the RNA solution 5 min at 65° C. in order to anneal primers to RNA, then the solution was kept on ice and split in two samples for RT (incubation 10 min 53° C. than 10 min 80° C.). Sample RT+: for 20 μL, add SSIV buffer 5×, 2 μL DTT, 2 μL Dnase inhibitor and 2 μL superscript RT. Sample RT−: for 10 μL, add SSIV buffer 5×, 1 μL DTT, 1 μL Dnase inhibitor and 1 μL superscript RT. 1/16th volume of E. coli RnaseH was added and the solution was incubated 20 min at 37° C. Primer annealing, RT and RNA digestion were performed with Invitrogen's SuperScript IV reagents.

### Ribonucleoprotein Complexes

The RNP complexes for targeting of the TRAC locus contained a TRAC sgRNA 5′-AGAGUCUCUCAGCUGGUACA-3′ (SEQ ID NO:107).

The RNP complexes for targeting of the BTK locus contained a BTK sgRNA 5′-GAUGCUCUCCAGAAUCACUG-3′ (SEQ ID NO:108).

### Transduction of T Cells, HSPCs and Murine Cells

T cells were electroporated with TRAC sgRNA/Cas9 RNP complexes as previously described by Wiebking et al. (2021, Haematologica, 106(3): 847-858), substituting the electroporation protocol from EO-115 to EO-100 (Seki and Rutz, 2018, Journal of Experimental Medicine, 215: 985-997).

HSPCs were electroporated with BTK sgRNA/Cas9 RNP complexes as previously described by Rai et al. (2020, Nature Communications, 11, Article Number: 4034) using the MaxCyte CTX Flow electroporator (MaxCyte, USA).

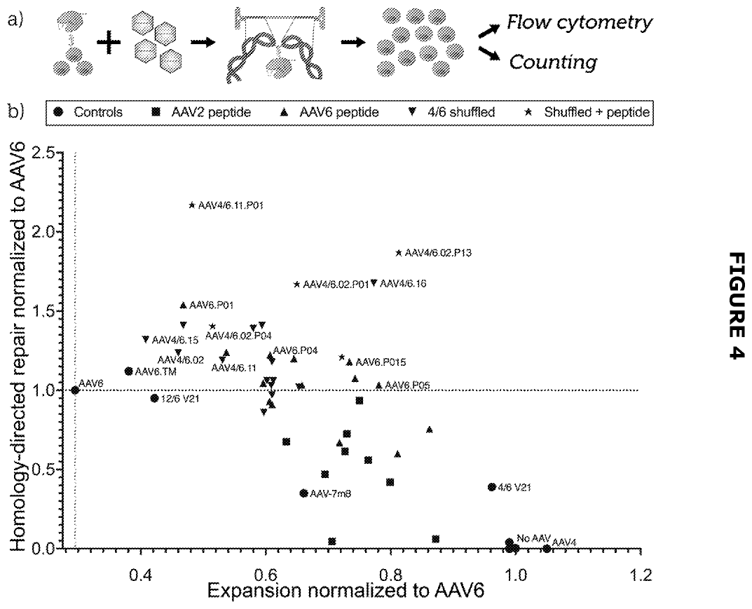

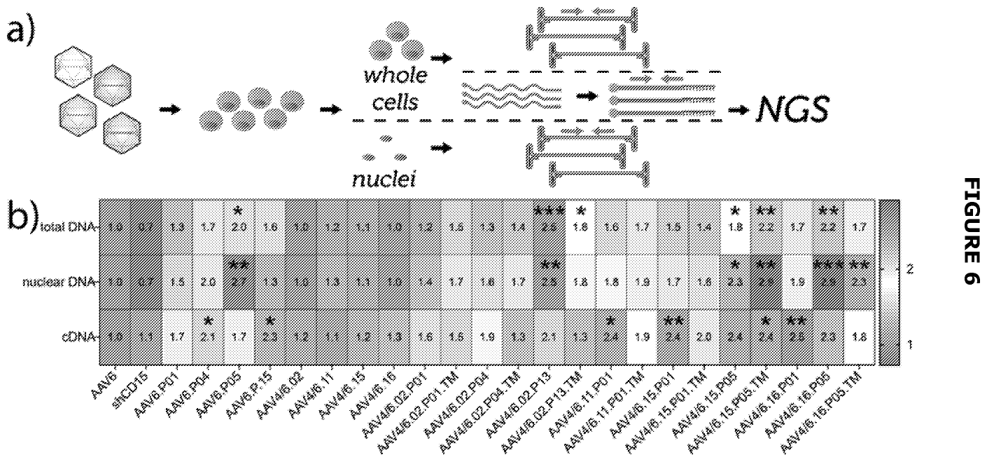

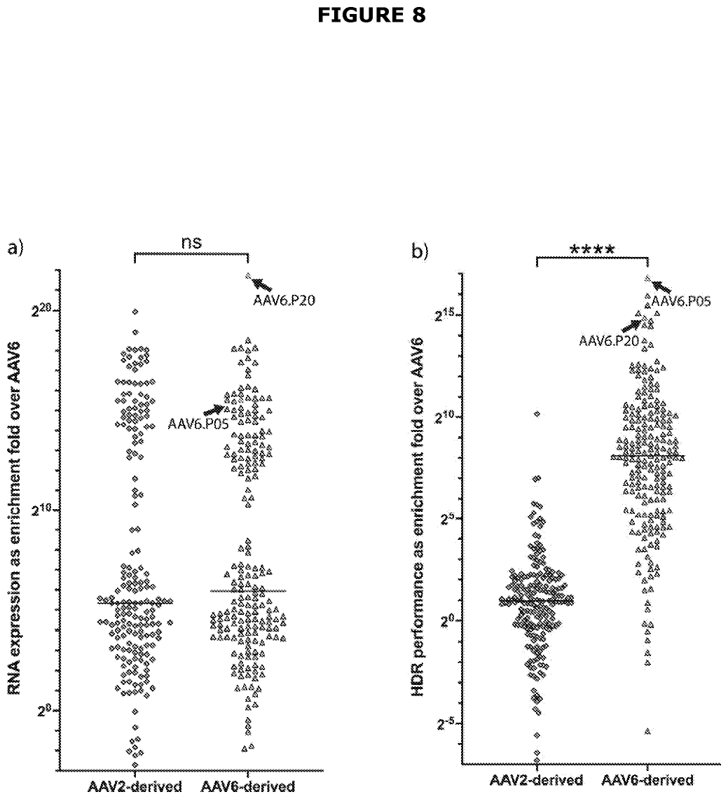

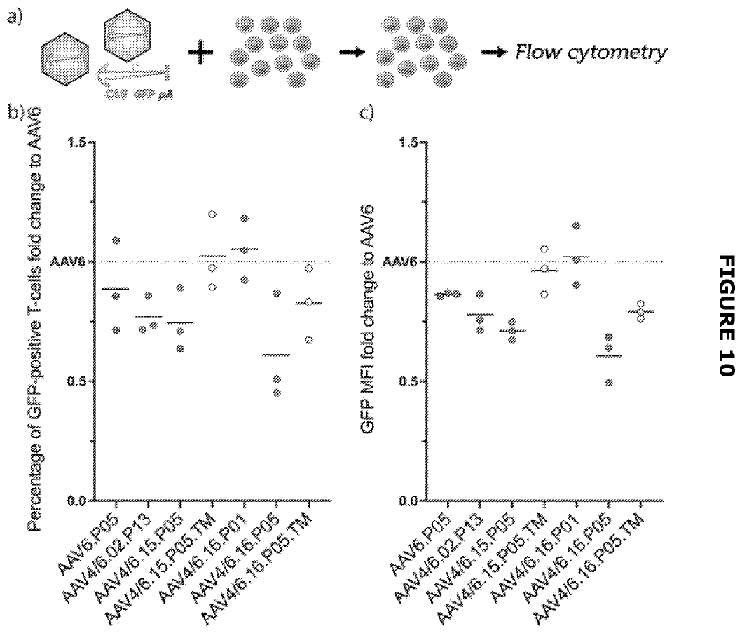

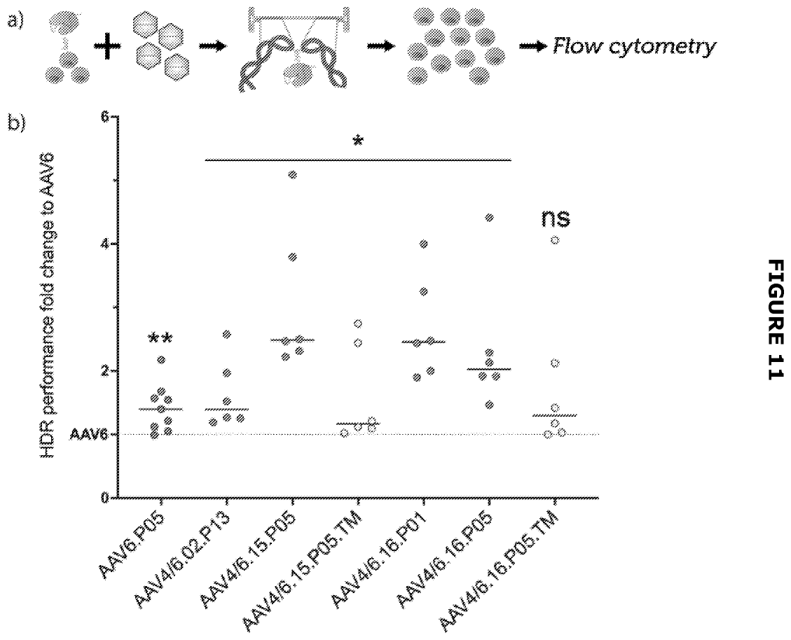

AAV capsids for individual testing (FIG. 4) were transduced at a dose of 10,000 vector genomes (vg) per cell (vg/cell). Validation mixes with CMV-driven barcoded GFP or homology arm-flanked barcoded GFP were transduced at a dose of 2,000 vg/cell (FIGS. 6-7 and 9-11). Transductions for follow-up validation were transduced at 1,000 vg/cell (FIG. 8).

### Genomic DNA Extraction

Two different methods to extract genomic DNA from T cells were used: a phenol-chloroform extraction (Westhaus et al., 2020, supra) and NEB HMW kit extraction (Cat #: T3050S).

Whole genome DNA was extracted using a phenol-chloroform method (Westhaus et al., 2020, supra) and long fragments were amplified from 200 ng of extracted DNA with one primer binding in the genomic region of either BTK (SEQ ID NO:101) or TRAC (SEQ ID NO:100) while the other primer bound the WPRE region of the barcoded transgene.

This PCR product was extracted using gel extraction and 10 ng were used for a second PCR for a small barcode resembling the one described by Kochanek (1999, Human Gene Therapy, 10(15): 2451-2459).

### Example 2. Selection of Capsids on the Basis of Homologous Recombination in T Cells

A study to selecting capsids on the basis of their ability to support homologous recombination in T cells was performed. The study was designed to use a two-round selection process, comprising an initial preselection process in which a capsid library was first screened using the functional transduction (FT) platform (as described in WO2020077411) to select for capsids that could facilitate functional transduction of T cells (i.e., transduction of, and transgene expression in, T cells). Selection using the homologous recombination (HR) platform was then performed, where capsids were selected on the basis of their ability to support homologous recombination in T cells.

For the first round (or preselection) process using the FT platform, T cells were transduced with three different libraries (AAV2 peptide library, AAV6 peptide library, AAV4/AAV6 shuffled library) packaged in the FT platform. The T cells were provided as buffy coat from the Australian Red Cross and isolated using either standard CD3 MACS isolation or pan-T-cell negative isolation. T cells were expanded in serum-free media and IL-2 supplement. Transduction of the libraries was performed at 10,000 vector genomes (vg) per cell (vg/cell). Cells were harvested three days after transduction and RNA was extracted using TRIZOL precipitation (Westhaus et al., 2020, supra). cDNA was generated using Superscript IV first-strand synthesis system. The AAV2 and AAV6 peptide regions were amplified using a forward primer (CTAACCCTGTGGCCACGG; SEQ ID NO:102) and reverse primer (CGTCTCTGTCTTGCCACACC; SEQ ID NO:103) primer to create the PCR peptide pool. The peptide pool was subsequently cloned into the background capsids in the TRAC or BTK HR platforms or the FT platform. Full-length shuffled capsids were amplified and inserted into a transfer plasmid before being excised and inserted into the TRAC or BTK HR platforms in preparation for the second round.

For the second round process, T cells were electroporated with sgRNA/Cas9 RNP complexes before being transduced with the three pre-selected libraries. Transduction occurred either 15 min, 2 hours or 4 hours after electroporation of the T cells. Following the transduction, the cells were expanded in IL2-containing media for 14 days before DNA extraction and recovery of peptide variants using in and out PCR. Full-lengths capsids were recovered and the peptide amplicon was further processed by cleavage using the MscI enzyme yielding a short DNA fragment compatible with NGS. The full length capsids were cleaved with SwaI allowing integration into a transfer plasmid and analysis of colonies to identify novel sequences by Sanger sequencing.

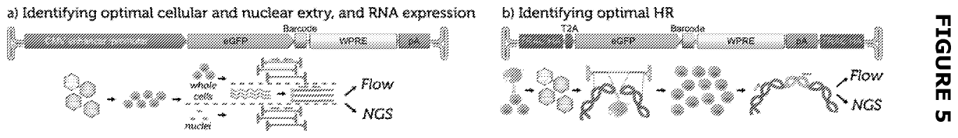

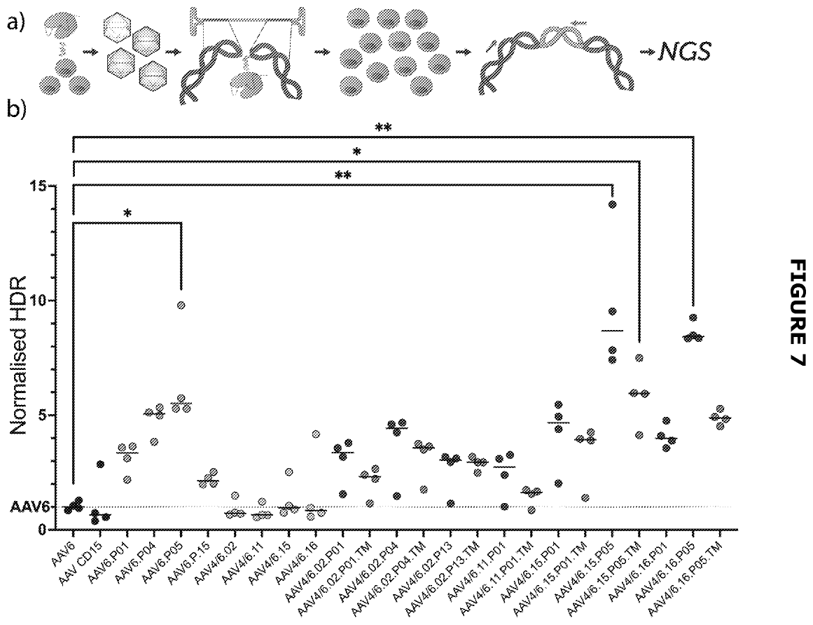

For validation of the selection process, and as shown in FIG. 5, a series of NGS-based technologies were used. Initially, the AAV capsids were tested for their ability to interact with cells (PCR from total DNA of T cells transduced with the AAV capsids), enter the nucleus (PCR from DNA in the nuclear fraction) and express RNA (which was converted into cDNA and PCR amplified) and AAV-mediated homology directed repair (HDR) by performing PCR with the primer binding sites inside the transgene (i.e., the barcoded reporter) and outside the homology arms. The results are shown in FIG. 6 and FIG. 7.

### Example 3. Performance of Novel AAV Capsids in T Cells

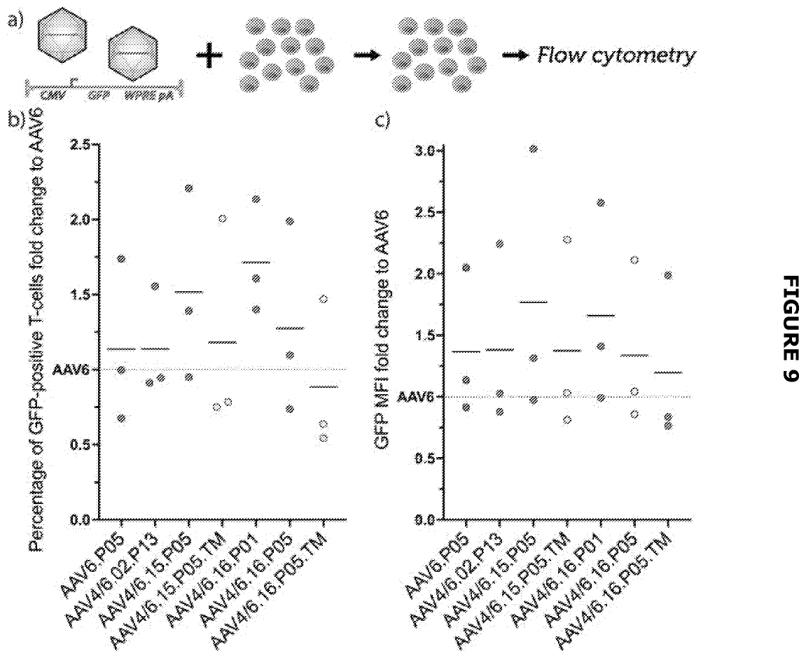

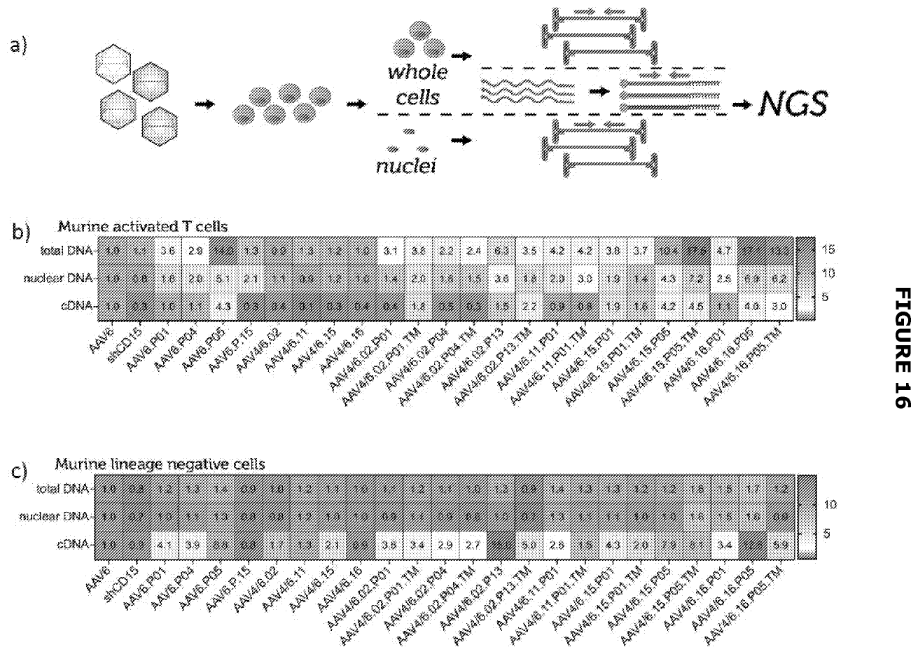

Of the three capsid libraries screened in T cells using the two-round selection process described above, a subset of novel AAV capsids were selected as having improved HDR-mediated gene editing in T cells (FIG. 8), noting that the enrichment based on RNA expression was not equivalent to enrichment based on HDR efficacy, with HDR enrichment favoring AAV6-derived variants. The performance of the top candidates from the selection process were confirmed by comparing the novel AAV capsids based on GFP integration in primary human T cells (FIGS. 9-11). The same novel AAV capsids also performed well in murine immune cells (see FIG. 16).

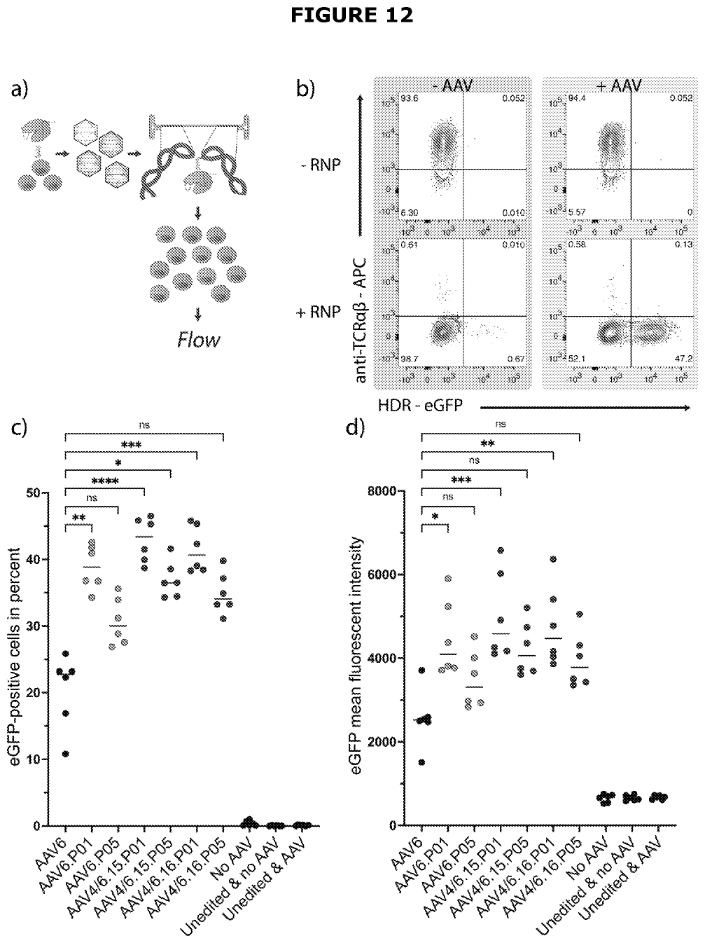

The inventors further evaluated the HDR performance of selected novel capsids in T cells (FIG. 12) using the methods described above. The novel capsids AAV6.P01, AAV6.P05, AAV4/6.15.P1, AAV4/6.15.P5, AAV4/6.16.P1 and AAV4/6.16.P5 are up to 2.3-fold more efficient at CRISPR/Cas9-mediated knock-in in primary T-cells than wild-type AAV6 (see FIG. 12 and Table 3 below).

TABLE 3
CRISPR/Cas9-mediated knock-in in primary T-cells (mean fold improvement compared to AAV6)

| AAV6 | AAV6.P01 | AAV6.P05 | AAV4/6.15.P1 | AAV4/6.15.P5 | AAV4/6.16.P1 | AAV4/6.16.P5 |
|---|---|---|---|---|---|---|
| 1 | 2.05 | 1.61 | 2.27 | 1.96 | 2.20 | 1.83 |

### Example 4. Performance of Novel AAV Capsids in HSPCs

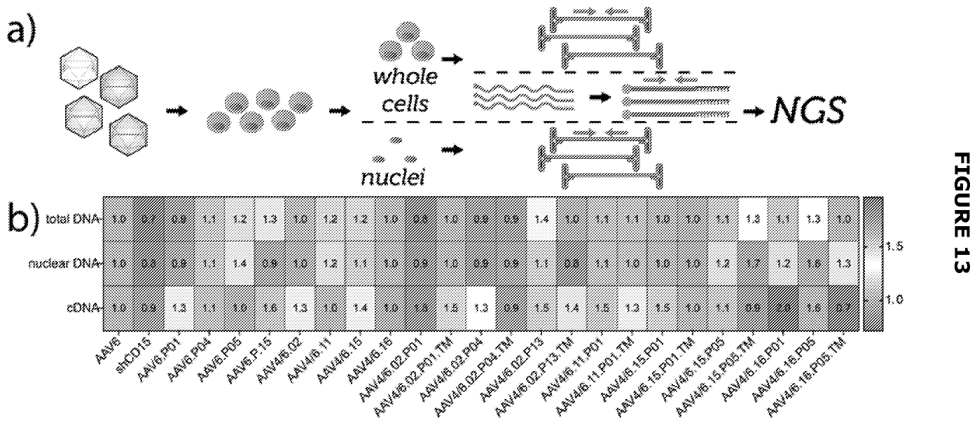

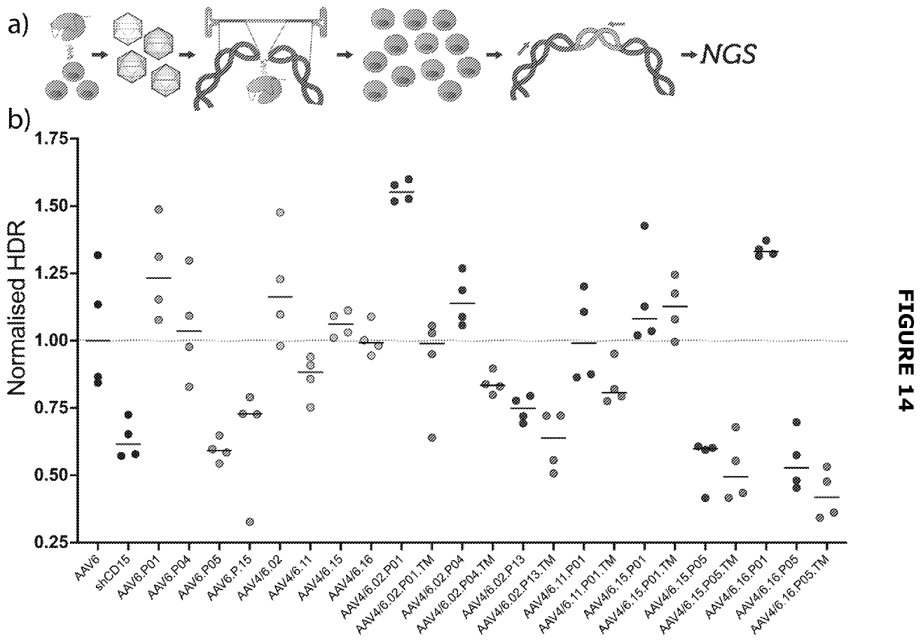

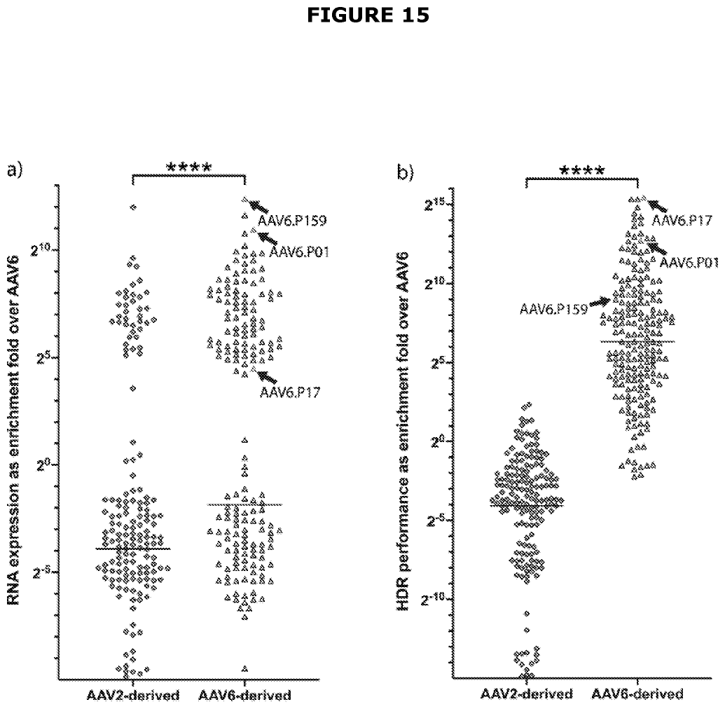

The best performing novel AAV capsids in human and murine immune cells were assessed for vector entry, nuclear translocation and transgene expression in HSPCs (FIG. 13). For this analysis, the homology arms were changed to correspond with the BTK locus. Interestingly, no improvement in gene editing of human HSPCs was shown (FIGS. 14 and 15).

1. An AAV capsid polypeptide, comprising a peptide modification relative to the AAV6 polypeptide set forth in SEQ ID NO:69, wherein the peptide modification comprises one or more or all of:
a) a peptide insertion in variable region 8 (VR-VIII);
b) 9 consecutive amino acids relative to the AAV6 polypeptide set forth in SEQ ID NO:69, comprising the sequence set forth in any one of SEQ ID NOs:70-97; and
c) a substitution of one or more amino acids in the region of the capsid polypeptide spanning positions 1-170 with numbering relative to SEQ ID NO:69, wherein the capsid polypeptide comprises about 85%, 86%, 87%, 88%, 89%, 90%, 91%, 92%, 93%, 94%, 95%, 96%, 97%, 98% or 99% sequence identity to positions 1-170 of SEQ ID NO:69, and wherein the substituted amino acid sequence is derived from the AAV4 polypeptide set forth in SEQ ID NO:109.

2. The AAV capsid polypeptide of claim 1, wherein the peptide insertion in variable region 8 (VR-VIII) is in the region of the capsid polypeptide spanning positions 581-593, with numbering relative to SEQ ID NO:69.

3. The AAV capsid polypeptide of claim 1 or claim 2, wherein the peptide modification comprises 9 consecutive amino acids, wherein 7 of those amino acids are insertions after position 588 relative to the AAV6 polypeptide set forth in SEQ ID NO:69.

4. The AAV capsid polypeptide of claim 1 or claim 2, the peptide modification is a substitution of one or more amino acids in the region of the capsid polypeptide spanning positions 15-165 with numbering relative to SEQ ID NO:69, wherein the capsid polypeptide comprises about 85%, 86%, 87%, 88%, 89%, 90%, 91%, 92%, 93%, 94%, 95%, 96%, 97%, 98% or 99% sequence identity to positions 15-165 of SEQ ID NO:69.

5. The AAV capsid polypeptide of any one of claims 1-4, comprising one or more amino acid substitutions at position 492, 705 and/or 731 relative to the AAV6 capsid polypeptide set forth in SEQ ID NO:69.

6. The AAV capsid polypeptide of claim 5, wherein the amino acid substitution is T492V, Y705F and/or Y731F relative to the AAV6 capsid polypeptide set forth in SEQ ID NO:69.

7. The AAV capsid polypeptide of any one of claims 1-6, comprising the sequence of amino acids set forth in any one of SEQ ID NOs:1-68, or a sequence having at least or about 85%, 86%, 87%, 88%, 89%, 90%, 91%, 92%, 93%, 94%, 95%, 96%, 97%, 98%, or 99% sequence identity thereto.

8. The AAV capsid polypeptide of claim 7, comprising the sequence of amino acids set forth in any one of SEQ ID NOs:1, 4, 49 and 58-61, or a sequence having at least or about 85%, 86%, 87%, 88%, 89%, 90%, 91%, 92%, 93%, 94%, 95%, 96%, 97%, 98%, or 99% sequence identity thereto.

9. An AAV capsid polypeptide, comprising:
a) a VP1 protein comprising the sequence of amino acids set forth in any one of SEQ ID NOs:1-68;
b) a VP2 protein comprised within the sequence of amino acids set forth in any one of SEQ ID NOs:1-68;
c) a VP3 protein comprised within the sequence of amino acids set forth in any one of SEQ ID NOs:1-68; or
d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c).

10. The AAV capsid polypeptide of claim 9, comprising:
a) a VP1 protein comprising the sequence of amino acids set forth in any one of SEQ ID NOs: 1, 4, 49 and 58-61;
b) a VP2 protein comprised within the sequence of amino acids set forth in any one of SEQ ID NOs: 1, 4, 49 and 58-61;
c) a VP3 protein comprised within the sequence of amino acids set forth in any one of SEQ ID NOs: 1, 4, 49 and 58-61; or
d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c).

11. An AAV vector comprising the capsid polypeptide of any one of claims 1-10.

12. The AAV vector of claim 11, wherein the vector further comprises a heterologous coding sequence.

13. The AAV vector of claim 12, wherein the heterologous coding sequence encodes a peptide, polypeptide or polynucleotide.

14. The AAV vector of claim 13, wherein the peptide, polypeptide or polynucleotide is a therapeutic peptide, polypeptide or polynucleotide.

15. The AAV vector of any one of claims 11 to 14, further comprising a left homology arm and a right homology arm, wherein the sequence of the left homology arm and the sequence of the right homology arm are homologous to sequences at a locus in the genomic DNA of a host cell.

16. A nucleic acid molecule encoding the AAV capsid polypeptide of any one of claims 1-10.

17. A vector comprising the nucleic acid molecule of claim 16.

18. The vector of claim 17, wherein the vector is selected from among a plasmid, cosmid, phage and transposon.

19. A host cell, comprising the AAV vector of any one of claims 11-15, the nucleic acid molecule of claim 16, or the vector of claim 17 or claim 18.

20. A method for introducing a heterologous coding sequence into a host cell, comprising contacting the host cell with the AAV vector of any one of claims 11-15.

21. The method of claim 20, further comprising a step of exposing the host cell to a genome editing nuclease before contacting the host cell with the AAV vector.

22. The method of claim 21, wherein the genome editing nuclease is selected from a zinc-finger nuclease (ZFN), transcription activator-like effector nucleases (TALEN) and clustered regularly interspaced short palindromic repeat (CRISPR)-Cas-associated nuclease.

23. The method of claim 21 or claim 22, wherein the step of exposing the host cell to a genome editing nuclease comprises exposing the host cell to a ribonucleoprotein complex comprising a CRISPR-Cas-associated nuclease and a guide RNA (gRNA).

24. The method of claim 22 or claim 23, wherein the CRISPR-Cas-associated nuclease is selected from a Cas3, Cas9, Cas12 (e.g., Cas12a, Cas12b, Cas12c, Cas12d, Cas12e) and Cas14.

25. The method of any one of claims 20-24, wherein the host cell is a T cell.

26. The method of claim 25, wherein the heterologous coding sequence encodes a chimeric antigen receptor (CAR).

27. The method of any one of claims 20-26, wherein contacting a host cell with the AAV vector comprises administering the AAV vector to a subject.

28. The method of any one of claims 21-27, wherein contacting a host cell with the AAV vector and genome editing nuclease comprises administering the AAV vector and genome editing nuclease to a subject.

29. The method of claim 27 or claim 28, wherein administration of the AAV vector or the AAV vector and genome editing nuclease to the subject effects the treatment of an immunodeficiency.

30. The method of claim 29, wherein the method is in vitro or ex vivo.

31. Use of the AAV vector of any one of claims 11-15 for the preparation of a medicament for treating an immunodeficiency.

32. The AAV vector of any one of claims 11-15 for use in the preparation of a medicament.

33. The AAV vector of claim 32, wherein the medicament is a medicament for treating an immunodeficiency.


SEQUENCE LISTING (109 sequences)

SEQ ID NO: 1 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSGDGDIKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEDMGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 4 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEAVEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEMEAKPEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 6 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSTNDDSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSNNISDKDQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDDQGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSVEGGEKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDVQGEKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSADDVTKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 12 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSNESEKKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSIETEPKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 14 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEGETGKKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDTADHKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQRSEKELEEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEGDAVDKKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 18 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSQTEGEKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 19 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSVETEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 20 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDANEKKEQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 21 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEDHADKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDDDNTKTKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 23 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDLDPDKIKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 24 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSTADDDKTKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 25 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSSEENTKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 26 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDDDNSKIKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDENNMKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 28 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSGKPEEKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 29 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 30 - protein, 736 aa, synthetic construct
  REGION 1..736: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 31 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEQAGETAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 32 - protein, 736 aa, synthetic construct
  REGION 1..736: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 33 - protein, 736 aa, synthetic construct
  REGION 1..736: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 34 - protein, 736 aa, synthetic construct
  REGION 1..736: note = Synthetic
MAADGYLPDWLEDNLSEGVREWWALQPGAPKPKANQQHQDNARGLVLPGYKYLGPGNGLDKGEPVNAADAAALEHDKAYDQQLKAGDNPYLRYNHADAEFQERLQEDTSFGGNLGRAVFQAKKRVLEPLGLVEQAGETAPGKKRPLIESPQQPDSSTGIGKKGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 35 - protein, 736 aa, synthetic construct
  REGION 1..736: note = Synthetic
MAADGYLPDWLEDNLSEGVREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 36 - protein, 736 aa, synthetic construct
  REGION 1..736: note = Synthetic
MAADGYLPDWLEDNLSEGIREWWDLKPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 37 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 38 - protein, 736 aa, synthetic construct
  REGION 1..736: note = Synthetic
MAADGYLPDWLEDNLSEGIREWWALQPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 39 - protein, 736 aa, synthetic construct
  REGION 1..736: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 40 - protein, 736 aa, synthetic construct
  REGION 1..736: note = Synthetic
MAADGYLPDWLEDNLSEGIREWWDLKPGAPKPKANQQKQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 41 - protein, 736 aa, synthetic construct
  REGION 1..736: note = Synthetic
MAADGYLPDWLEDNLSEGVREWWALQPGAPKPKANQQHQDDARGLVLPGYKYLGPFNGLDKGEPVNAADAAALEHDKAYDQQLKAGDNPYLKYNHADAEFQERLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 736 aa, synthetic construct
  REGION 1..736: note = Synthetic
MAADGYLPDWLEDNLSEGVREWWGLQPGAPKPKANQQHQDNARGLVLPGYKYLGPFNGLDKGEPVNAADAAALEHDKAYDQQLKAGDNPYLRYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 43 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 44 - protein, 736 aa, synthetic construct
  REGION 1..736: note = Synthetic
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 45 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 46 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSGDGDIKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEDMGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDGVAKDEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 49 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSNNISDKDQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 50 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDDQGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 51 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 52 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSGDGDIKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 53 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSVSESHKEQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 54 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEDMGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 55 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDGVAKDEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 56 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSNNISDKDQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 57 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSDDQGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 58 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 59 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEAVEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 743 aa, synthetic construct
  REGION 1..743: note = Synthetic
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 61 - protein, 743 aa, synthetic construct
  REGION 1..743: note = Synthetic
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQGQSEAVEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 62 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 63 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEDMGSKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 64 - protein, 743 aa, synthetic construct
  REGION 1..743: note = Synthetic
MAADGYLPDWLEDNLSEGVREWWALQPGAPKPKANQQHQDNARGLVLPGYKYLGPFNGLDKGEPVNAADAAALEHDKAYDQQLKAGDNPYLKYNHADAEFQQRLQGDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSNNISDKDQAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 65 - protein, 743 aa, synthetic construct
  REGION 1..743: note = Synthetic
MAADGYLPDWLEDNLSEGVREWWALQPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 66 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEEVGGKDKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 67 - protein, 743 aa, synthetic construct
  REGION 1..743: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEAVEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 68 - protein, 743 aa, synthetic construct
  REGION 1..743: note = Synthetic
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKVKTDNNNSNFTWTGASKYNLNGRESIINPGTAMASHKDDKDKFFPMSGVMIFGKESAGASNTALDNVMITDEEEIKATNPVATERFGTVAVNLQGQSEAVEGKEKAAATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNFAKSANVDFTVDNNGLYTEPRPIGTRFLTRPL

SEQ ID NO: 69 - protein, 736 aa, Adeno-associated virus, serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 70 - protein, 9 aa, synthetic construct
  REGION 1..9: note = Synthetic
RSEKELEEK

SEQ ID NO: 71 - protein, 9 aa, synthetic construct
  REGION 1..9: note = Synthetic
SEEGETGKK

SEQ ID NO: 72 - protein, 9 aa, synthetic construct
  REGION 1..9: note = Synthetic
SNNISDKDQ

SEQ ID NO: 73 - protein, 9 aa, synthetic construct
  REGION 1..9: note = Synthetic
SADDVTKDK

SEQ ID NO: 74 - protein, 9 aa, synthetic construct
  REGION 1..9: note = Synthetic
SDDDNTKTK

SEQ ID NO: 75 - protein, 9 aa, synthetic construct
  REGION 1..9: note = Synthetic
SDDDNSKIK

SEQ ID NO: 76 - protein, 9 aa, synthetic construct
  REGION 1..9: note = Synthetic
SDENNMKDK

SEQ ID NO: 77 - protein, 9 aa, synthetic construct
  REGION 1..9: note = Synthetic
SEDHADKDK

SEQ ID NO: 78 - protein, 9 aa, synthetic construct
  REGION 1..9: note = Synthetic
SDANEKKEQ

SEQ ID NO: 79 - protein, 9 aa, synthetic construct
  REGION 1..9: note = Synthetic
SEEVGGKDK

SEQ ID NO: 80 - protein, 9 aa, synthetic construct
  REGION 1..9: note = Synthetic
SEAVEGKEK

SEQ ID NO: 81 - protein, 9 aa, synthetic construct
  REGION 1..9: note = Synthetic
SNESEKKEK

SEQ ID NO: 82 - protein, 9 aa, synthetic construct
  REGION 1..9: note = Synthetic
SIETEPKEK

SEQ ID NO: 83 - protein, 9 aa, synthetic construct
  REGION 1..9: note = Synthetic
SVETEGKEK

SEQ ID NO: 84 - protein, 9 aa, synthetic construct
  REGION 1..9: note = Synthetic
SEGDAVDKK

SEQ ID NO: 85 - protein, 9 aa, synthetic construct
  REGION 1..9: note = Synthetic
SEMEAKPEK

SEQ ID NO: 86 - protein, 9 aa, synthetic construct
  REGION 1..9: note = Synthetic
STNDDSKEK

SEQ ID NO: 87 - protein, 9 aa, synthetic construct
  REGION 1..9: note = Synthetic
STADDDKTK

SEQ ID NO: 88 - protein, 9 aa, synthetic construct
  REGION 1..9: note = Synthetic
SGKPEEKEK

SEQ ID NO: 89 - protein, 9 aa, synthetic construct
  REGION 1..9: note = Synthetic
SSEENTKEK

SEQ ID NO: 90 - protein, 9 aa, synthetic construct
  REGION 1..9: note = Synthetic
SDTADHKEK

SEQ ID NO: 91 - protein, 9 aa, synthetic construct
  REGION 1..9: note = Synthetic
SGDGDIKEK

SEQ ID NO: 92 - protein, 9 aa, synthetic construct
  REGION 1..9: note = Synthetic
SQTEGEKEK

SEQ ID NO: 93 - protein, 9 aa, synthetic construct
  REGION 1..9: note = Synthetic
SVEGGEKEK

SEQ ID NO: 94 - protein, 9 aa, synthetic construct
  REGION 1..9: note = Synthetic
SDVQGEKEK

SEQ ID NO: 95 - protein, 9 aa, synthetic construct
  REGION 1..9: note = Synthetic
SEDMGSKEK

SEQ ID NO: 96 - protein, 9 aa, synthetic construct
  REGION 1..9: note = Synthetic
SDDQGSKEK

SEQ ID NO: 97 - protein, 9 aa, synthetic construct
  REGION 1..9: note = Synthetic
SDLDPDKIK

SEQ ID NO: 98 - DNA, 20 bp, synthetic construct
  misc_feature 1..20: note = Synthetic
  source 1..20: mol_type = other DNA
tcaagatccgccacaacatc

SEQ ID NO: 99 - DNA, 20 bp, synthetic construct
  misc_feature 1..20: note = Synthetic
  source 1..20: mol_type = other DNA
ttctcgttggggtctttgct

SEQ ID NO: 100 - DNA, 26 bp, synthetic construct
  misc_feature 1..26: note = Synthetic
  source 1..26: mol_type = other DNA
gccagagttatattgctggggttttg

SEQ ID NO: 101 - DNA, 24 bp, synthetic construct
  misc_feature 1..24: note = Synthetic
  source 1..24: mol_type = other DNA
ggagtattgtaggtttggggaggc

SEQ ID NO: 102 - DNA, 18 bp, synthetic construct
  misc_feature 1..18: note = Synthetic
  source 1..18: mol_type = other DNA
ctaaccctgtggccacgg

SEQ ID NO: 103 - DNA, 20 bp, synthetic construct
  misc_feature 1..20: note = Synthetic
  source 1..20: mol_type = other DNA
cgtctctgtcttgccacacc

SEQ ID NO: 104 - DNA, 22 bp, synthetic construct
  misc_feature 1..22: note = Synthetic
  source 1..22: mol_type = other DNA
attggctcgaggacaacctctc

SEQ ID NO: 105 - DNA, 35 bp, synthetic construct
  misc_feature 1..35: note = Synthetic
  source 1..35: mol_type = other DNA
ttaaacggtttattgattaacaggcaattacaggg

SEQ ID NO: 106 - DNA, 36 bp, synthetic construct
  misc_feature 1..36: note = Synthetic
  source 1..36: mol_type = other DNA
ttaaacggtttattgattaacaagcaattacaggtg

SEQ ID NO: 107 - RNA, 20 nt, synthetic construct
  misc_feature 1..20: note = Synthetic
  source 1..20: mol_type = other RNA
agagtctctcagctggtaca

SEQ ID NO: 108 - RNA, 20 nt, synthetic construct
  misc_feature 1..20: note = Synthetic
  source 1..20: mol_type = other RNA
gatgctctccagaatcactg

SEQ ID NO: 109 - protein, 734 aa, Adeno-associated virus, serotype 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL